(19) United States
(12) Patent Application Publication
(10) Pub. No.: US 2024/0212824 A1
(43) Pub. Date: Jun. 27, 2024

(54) ADMINISTERING A DIGITAL THERAPEUTIC USING A DEVICE INTERFACE TO TREAT ANXIETY OR DEPRESSION

(71) Applicant: Woebot Labs, Inc., San Francisco, CA (US)

(72) Inventors: Alison Darcy, Dublin (IE); Jade Daniels, San Francisco, CA (US); Athena Robinson, San Carlos, CA (US); Casey Sackett, Corte Madera, CA (US)

(21) Appl. No.: 18/393,531

(22) Filed: Dec. 21, 2023

Related U.S. Application Data
(60) Provisional application No. 63/434,461, filed on Dec. 21, 2022.
(60) Provisional application No. 63/434,462, filed on Dec. 21, 2022.
(60) Provisional application No. 63/434,469, filed on Dec. 22, 2022.

(51) Int. Cl. G16H 20/70; A61B 5/16; G16H 80/00

(52) U.S. Cl. CPC G16H 20/70; A61B 5/165; G16H 80/00

(57) ABSTRACT

Methods, systems, and computer programs are described for treating anxiety or depression. The method includes receiving a notification that indicates that a user is to begin a therapeutic session, triggering a therapeutic session based on the received notification, wherein triggering the therapeutic session includes creating a messaging session between the user and a conversation engine of a therapeutic system, aggregating a conversation thread based on communications received from the conversation engine and responses to communications received from the conversation engine input by a user of the user device, providing response data, to the therapeutic system, that includes the conversation thread, obtaining, from the therapeutic system, therapeutic content based on the response data that treats one or more symptoms of anxiety or depression exhibited by the user, and administering a therapeutic treatment to the user using the obtained digital content a predetermined number of times a week for a predetermined duration.

### CROSS-REFERENCE TO RELATED APPLICATION

This application claims the benefit of U.S. Provisional Patent Application No. 63/434,461 titled “Administering A Digital Therapeutic Using A Device Interface To Treat Anxiety or Depression,” U.S. Provisional Patent Application No. 63/434,462 titled “Administering A Digital Therapeutic Using A Device Interface To Treat Stress,” and U.S. Provisional Patent Application No. 63/434,469 titled “Administering A Digital Therapeutic Using A Device Interface To Treat Postpartum Depression,” each of which is herein incorporated by reference in their entirety.

### BACKGROUND

Digital mental health interventions (DMHIs) may include interactive software applications configured to treat symptoms of anxiety and/or depression. DMHIs are beneficial because they are designed to reduce treatment access issues for those experiencing those symptoms.

### SUMMARY

According to one innovative aspect of the present disclosure, a method for treating anxiety or depression is disclosed. In one aspect, the method can include actions of receiving, by a user device and from a therapeutic system, a notification that indicates that a user is to begin a therapeutic session, triggering, by the user device, a therapeutic session based on the received notification, wherein triggering the therapeutic session includes creating of a messaging session between the user and a conversation engine of the therapeutic system, aggregating, by the user device, a conversation thread based on communications received from the conversation engine and responses to communications received from the conversation engine input by a user of the user device, providing, by the user device, response data, to the therapeutic system, that includes the conversation thread, obtaining, by the user device and from the therapeutic system, therapeutic content based on the response data that, when rendered, by the user device and consumed by a user, treats one or more symptoms of anxiety or depression exhibited by the user, and administering a therapeutic treatment, by the user device, to the user using the obtained digital content, wherein administering the treatment comprises rendering, by the user device, the obtained digital content on a graphical user interface when the user is viewing the graphical user interface, a predetermined number of times a week for a predetermined duration.

Other versions include corresponding systems, apparatus, and computer programs to perform the actions of methods defined by instructions encoded on computer readable storage devices.

These and other versions may optionally include one or more of the following features. For instance, in some implementations, the conversation thread can include at least one communication from the conversation engine and at least one communication from the user that is a response to the communication from the conversation engine.

In some implementations, the conversation thread can include (i) a single natural language communication from the conversation engine and (ii) a single natural language response from the user.

In some implementations, the conversation thread can include (i) multiple natural language communications from the conversation engine and (ii) multiple natural language responses from the user.

In some implementations, the conversation thread can include (i) a plurality of pre-programmed graphical elements, with each of the pre-programmed graphical elements corresponding to an emotional state of the user and (ii) response data indicating selection of one or more pre-programmed graphical elements by the user.

In some implementations, the method for treating anxiety or depression can further include receiving, by the user device and from the therapeutic system, a request for feedback following completion of a therapeutic session, providing, by the user device, feedback data entered by the user to the therapeutic system, and receiving, by the user device, instructions to terminate the administered treatment prior to the completion of the full duration of the treatment prescribed by the therapeutic system.

In some implementations, obtaining, by the user device and from the therapeutic system, therapeutic content based on the response data that, when rendered, by the user device and consumed by a user, treats one or more symptoms of anxiety or depression exhibited by the user can include obtaining, by the user device, one or more interpersonal psychotherapy (IPT) tools and one or more cognitive behavioral therapy (CTB) tools based on the response data.

In some implementations, administering a therapeutic treatment, by the user device, to the user using the obtained digital content can include rendering, by the user device, one or more interpersonal psychotherapy (IPT) tools and one or more cognitive behavioral therapy (CBT) tools in the user interface of the user device a predetermined number of times a week for a duration of two to eight weeks.

In some implementations, administering a therapeutic treatment, by the user device, to the user using the obtained digital content can include using the user device to deliver interactive cognitive behavioral therapy (CBT) that comprises natural language interaction with a conversation engine.

In some implementations, the natural language interaction comprises (i) one or more natural language communications from the conversation engine to the user of the user device and (ii) one or more natural language responses from the user of the user device to the conversation engine.

In some implementations, administering a therapeutic treatment, by the user device, to the user using the obtained digital content can include using the user device to deliver an interactive therapy that comprises natural language interaction with a conversation engine.

In some implementations, the natural language interaction comprises (i) one or more natural language communications from the conversation engine to the user of the user device and (ii) one or more natural language responses from the user of the user device to the conversation engine.

In some implementations, the predetermined number of times a week comprises 3 to 5 times per week.

In some implementations, the predetermined number of times a week comprises daily use during the week.

In some implementations, the predetermined duration is two to eight weeks.

In some implementations, the predetermined duration is at least 8 weeks.

In some implementations, the method for treating anxiety or depression comprises a method for treating clinical levels of anxiety or depression symptoms.

In some implementations, the therapeutic content is administered for at least 5 minutes during each of the predetermined number of times per week.

In some implementations, the therapeutic content is administered at least 10 minutes during each of the predetermined number of times per week.

In some implementations, the predetermined number of times a week for a predetermined duration comprises at least 4 weeks of an 8-week time period.

In some implementations, anxiety or depression comprises postpartum depression.

According to another innovative aspect of the present disclosure, a method for treating stress or burnout is disclosed. In one aspect, the method can include actions of receiving, by a user device and from a therapeutic system, a notification that indicates that a user is to begin a therapeutic session, triggering, by the user device, a therapeutic session based on the received notification, wherein triggering the therapeutic session includes creating of a messaging session between the user and a conversation engine of the therapeutic system, aggregating, by the user device, a conversation thread based on communications received from the conversation engine and responses to communications received from the conversation engine input by a user of the user device, providing, by the user device, response data, to the therapeutic system, that includes the conversation thread, obtaining, by the user device and from the therapeutic system, therapeutic content based on the response data that, when rendered, by the user device and consumed by a user, treats one or more symptoms of stress or burnout exhibited by the user, and administering a therapeutic treatment, by the user device, to the user using the obtained digital content, wherein administering the treatment comprises rendering, by the user device, the obtained digital content on a graphical user interface when the user is viewing the graphical user interface, a predetermined number of times a week for a predetermined duration.

Other versions include corresponding systems, apparatus, and computer programs to perform the actions of methods defined by instructions encoded on computer readable storage devices.

These and other versions may optionally include one or more of the following features. For instance, in some implementations, the conversation thread can include at least one communication from the conversation engine and at least one communication from the user that is a response to the communication from the conversation engine.

In some implementations, the conversation thread can include (i) a single natural language communication from the conversation engine and (ii) a single natural language response from the user.

In some implementations, the conversation thread can include (i) multiple natural language communications from the conversation engine and (ii) multiple natural language responses from the user.

In some implementations, the conversation thread can include (i) a plurality of pre-programmed graphical elements, with each of the pre-programmed graphical elements corresponding to an emotional state of the user and (ii) response data indicating selection of one or more pre-programmed graphical elements by the user.

In some implementations, the method for treating stress or burnout can further include receiving, by the user device and from the therapeutic system, a request for feedback following completion of a therapeutic session, providing, by the user device, feedback data entered by the user to the therapeutic system, and receiving, by the user device, instructions to terminate the administered treatment prior to the completion of the full duration of the treatment prescribed by the therapeutic system.

In some implementations, obtaining, by the user device and from the therapeutic system, therapeutic content based on the response data that, when rendered, by the user device and consumed by a user, treats one or more symptoms of stress or burnout exhibited by the user can include obtaining, by the user device, one or more interpersonal psychotherapy (IPT) tools and one or more cognitive behavioral therapy (CTB) tools based on the response data.

In some implementations, administering a therapeutic treatment, by the user device, to the user using the obtained digital content can include rendering, by the user device, one or more interpersonal psychotherapy (IPT) tools and one or more cognitive behavioral therapy (CBT) tools in the user interface of the user device a predetermined number of times a week for a duration of two to eight weeks.

In some implementations, administering a therapeutic treatment, by the user device, to the user using the obtained digital content can include using the user device to deliver interactive cognitive behavioral therapy (CBT) that comprises natural language interaction with a conversation engine.

In some implementations, the natural language interaction comprises (i) one or more natural language communications from the conversation engine to the user of the user device and (ii) one or more natural language responses from the user of the user device to the conversation engine.

In some implementations, administering a therapeutic treatment, by the user device, to the user using the obtained digital content can include using the user device to deliver an interactive therapy that comprises natural language interaction with a conversation engine.

In some implementations, the natural language interaction comprises (i) one or more natural language communications from the conversation engine to the user of the user device and (ii) one or more natural language responses from the user of the user device to the conversation engine.

In some implementations, the predetermined number of times a week comprises 3 to 5 times per week.

In some implementations, the predetermined number of times a week comprises daily use during the week.

In some implementations, the predetermined duration is two to eight weeks.

In some implementations, the predetermined duration is at least 8 weeks.

In some implementations, the method for treating stress or burnout comprises a method for treating a method for treating clinically elevated levels of stress or burnout.

In some implementations, the therapeutic content is administered for at least 5 minutes during each of the predetermined number of times per week.

In some implementations, the therapeutic content is administered at least 10 minutes during each of the predetermined number of times per week.

In some implementations, the predetermined number of times a week for a predetermined duration comprises at least 4 weeks of an 8-week time period.

These and other innovative aspects of the present disclosure are readily apparent in view of the detailed description, the accompanying drawings, and the claims.

### BRIEF DESCRIPTION OF THE DRAWINGS

FIG. 1 is a diagram of an example of a system for generating, dosing, and administering a digital therapeutic to treat one or more symptoms related to anxiety or depression.

FIG. 2 is a flowchart of an example of a process that can be executed by a diagnosis engine, in accordance with one aspect of the present disclosure.

FIG. 3 is a flowchart of an example of a process that can be executed by a dosing engine, in accordance with one aspect of the present disclosure.

FIG. 4 is a flowchart of an example of a process that can be executed by a treatment engine, in accordance with one aspect of the present disclosure.

FIG. 5 is a flowchart of an example of a process that can executed by a persistent symptom mitigation engine, in accordance with one aspect of the present disclosure.

FIG. 6 is a diagram of an example of a user device that is configured to generate, dose, and administer a digital therapeutic to treat one or more symptoms related to anxiety or depression.

FIG. 7 is flowchart of an example of a process for administering a digital therapeutic for treating anxiety or depression, in accordance with one aspect of the present disclosure.

FIG. 8 is a flowchart of an example of a process for administering a digital therapeutic for treating stress or burnout, in accordance with one aspect of the present disclosure.

FIG. 9 is a block diagram of system components that can be used to implement a system for generating, dosing, and administering a digital therapeutic to treat one or more symptoms related to anxiety or depression.

FIG. 10 is a CONSORT diagram describing participant selection and participate in a study that used the present disclosure to treat anxiety or depression.

### DETAILED DESCRIPTION

The present disclosure is directed towards methods, systems, and computer programs for treating one or more symptoms of anxiety or depression for patients diagnosed with depression and/or anxiety, or for users with symptoms of depression or anxiety. A symptom of anxiety or depression can include, for example, a mental or physical feature of an individual which is regarded as indicating a presence of anxiety or depression. In some implementations, the present disclosure is configured to treat one or more symptoms of anxiety or depression using particularly configured digital mental health interventions (DMHIs) that are administered to an individual during a prescribed or recommended dosing schedule. An example of a DMHI can include, for example, administering a digital therapeutic to the user a prescribed number of times during a prescribed time period. In one implementation, the prescribed dosing schedule is one or more times per week for two to eight weeks. As described herein, “prescribed” may relate to prescription by a health care provider, or recommendation by the DMHI to the user. In some implementations, a DMHI is performed using the system 100 or user device 610.

In some implementations, the DMHI can occur by using a conversational agent to optimize engagement with the user (e.g., a patient diagnosed as exhibiting one or more symptoms of anxiety or depression). The conversational agent can include, for example, a conversational engine that is configured to engage in natural language communication with the user. Engaging in natural language communication with the user can include, for example, using a natural language processor for deciphering natural language inputs received from the user, generating a natural language response to the user based on the received natural language input, and communicating the generated natural language response to the user.

Multiple benefits are achieved by use of the conversational agent, as the conversational agent is configured to communicate with the user conversationally using the conversation engine in a manner that exhibits a friendly “personality” in an effort to establish a relationship, or other alliance, with the user. The conversational agent is designed to use this relationship to cause the user to developed a therapeutic alliance with the conversational agent, engage the virtual entity in a manner that provides characterizations of, e.g., the user's mood, and then to adhere to the treatment ultimately administered to the user via the virtual entity based on, e.g., the user's mood, as deciphered by the virtual entity's natural language processing performed by the virtual entity's conversation engine, mood evaluation engine, or both. For instance, the virtual entity may utilized BERT, transformers, or other NLP models to analyze a user's natural language responses, and identify problems or issues to address.

Additional benefits can be achieved by implementation of a persistent symptom mitigation engine. The persistent symptom mitigation engine dynamically measure an individual's current mood, changes in the individual's mood, or trends in the changes of the individual's mood after treatment of one or more symptoms of anxiety or depression has begun. For example, the persistent symptom mitigation engine can evaluate feedback (for instance in a natural language response), from an individual, after the administration of treatment of a digital therapeutic. Then, based on the evaluation of this received feedback, the persistent symptom mitigation engine can determine an individual's current mood, changes in the individual's mood, or trends in the changes of the individual's mood. This dynamic measurement of the level of one or more symptoms yields clinical and technological benefits.

The clinical benefits of this dynamic measurement include the dynamic adjustment of a treatment or dosing schedule to be made based on the individual's response to an initial treatment. Such benefits enable a digital therapeutic or a dosing schedule for the digital therapeutic to be dynamically customized based on an individual's responsiveness to an initial treatment. This dynamically customized treatment and/or dosing schedule helps to improve the medical outcomes of an individual being treated using the digital therapeutic of the present disclosure, as the treatment and/or dosing schedule is tuned to the precise mental and/or emotional state of an individual.

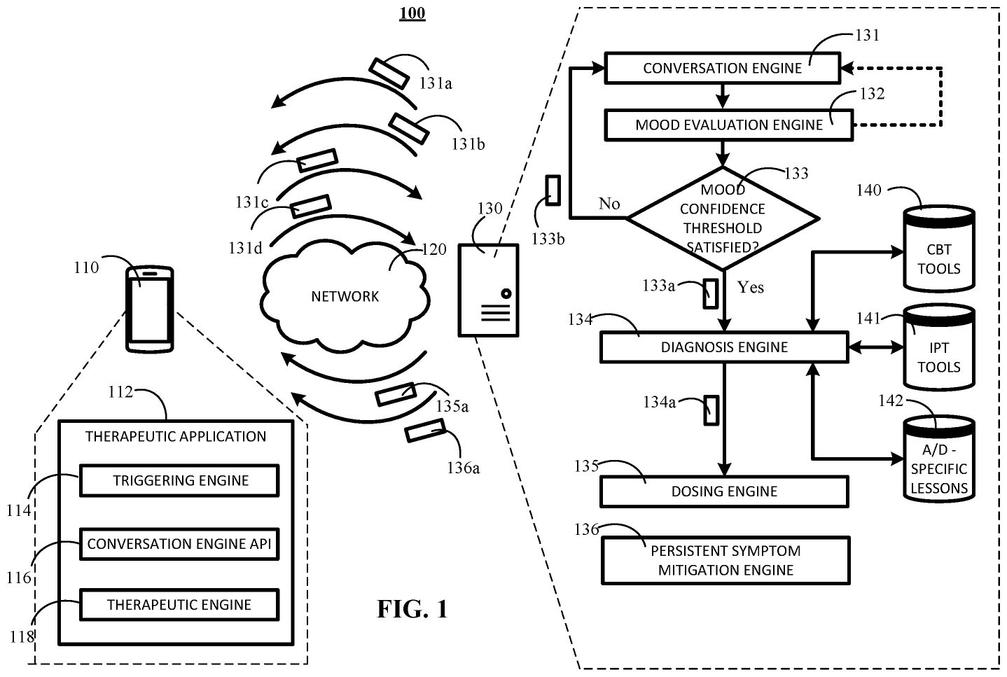

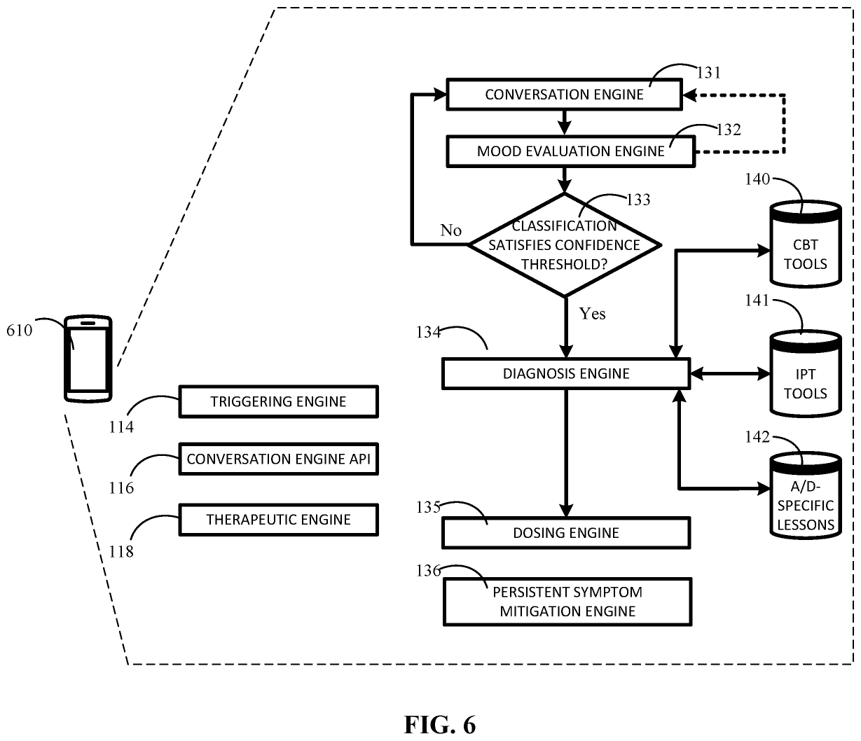

The technological benefits of this dynamic measurement includes the conservation of resources that is achieved by using the persistent symptom mitigation engine. In particular, persistent symptom mitigation engine is capable of terminating administration of a digital therapeutic treatment before the end of a prescribed therapeutic regimen if the persistent system mitigation engine determines that the user current mood, change in mood, or trend in mood change indicates that an individual's symptoms of anxiety or depression have been sufficiently mitigated. When administration of a treatment of a digital therapeutic is terminated before the end of a prescribed therapeutic regimen, the system of the present disclosure conserves the resources that would have been spent to administer the digital therapeutic for the remainder of the therapeutic regimen, thus achieving a technical improvement to the computing system. Specifically, there will be a reduction in the amount of processor (e.g., CPU, GPU, etc.) cycles executed, memory resources utilized, and bandwidth utilized when administration of the digital therapeutic is terminated earlier than a prescribed therapeutic regimen. This reduction in resources is achieved on the server side (e.g., server 130 does not cause the user device 110 to continue administering treatment at stage 440), on the user device side (e.g., user device 110 does not continue administering treatment at stage 440), or both. In some instances, where the present disclosure is fully implemented on the user device 110 such as in FIG. 6 (as opposed to the split user device side/server side architecture of FIG. 1), the user device 110 realizes a reduction in resources in both of the aforementioned scenarios.

In addition to the benefits and/or technical improvements identified above, there is a need, in the art, for the administration of such digital therapeutics. For example, an individual may be unable to take prescription medicine for depression or anxiety due to side effects, allergies, or other reasons. In such instances, methods of the present disclosure can meet this need as an alternative treatment for individuals who may not have access to therapists, and/or may be unwilling to engage in therapy with a live person.

FIG. 1 is a diagram of an example of a system 100 for generating, dosing, and administering a digital therapeutic to treat one or more symptoms of anxiety or depression.

The system 100 can include a user device 110, a network 120, and an application server 130. The user device 110 can include a smartphone, tablet computer, a laptop computer, a desktop computer, a smart television, a smartwatch, or the like. The user device 110 can include therapeutic application 112 that is installed, thereon. The therapeutic application 112 can include a treatment triggering engine 114, a conversation engine API 116, and a therapeutic engine 118. The network 120 can include one or more of a wired or wireless local area network, a short-wave radio network, a cellular network, the Internet, or any combination thereof. The application server 130 can include one or more computers configured to store and execute the respective engines 131, 132, 133, 134, 135, 136, shown in FIG. 1. In some implementations as shown in FIG. 1, the databases 140, 141, 142 may be stored within one or more computers of the application server 130. However, in other implementations, the databases 140, 141, 142 may be hosted by one or more remote computers. In such implementations, application server 130 can be configured to interact with the databases 140, 141, 142 by communicating with the one or more remote computers. In the example of FIG. 1, the databases 140, 141, 142 include lessons that can be selected and provided to a user device 110 as part of a digital therapeutic configured to treat one or more symptoms of anxiety or depression.

With reference to FIG. 1, the therapeutic application 112 is installed on the user device 110. Then, at some point in time after installation of the therapeutic application 112 on the user device 110, execution of the system 100 begins with the triggering engine 114 triggering a therapeutic treatment session. A therapeutic treatment session can be described as the time period that begins with the therapeutic application 112 initiating interaction with an individual (e.g., user of user device 110) through either (a) completion of a therapeutic treatment or (b) a determination, by the therapeutic application, that the individual does not need a therapeutic treatment. For purposes of this disclosure, “individual” is understood to be the “user” of the user device 110 that is being receiving treatment for one or more symptoms of a anxiety or depression from the therapeutic application. Accordingly, the words “individual” and “user” are intended to have the same meaning and may be used interchangeably.

The triggering engine 114 is configured to drive adherence of a user to a therapeutic treatment for one or more symptoms of anxiety or depression. The trigger engine 114 achieves this by autonomously initiating interactions with the user of the user device 110 to determine an emotional state of the user. In some implementations, initiating interaction with the user can include, for example, receiving notifications 131a (e.g., push notifications) and generating alerts for output by the user device 110. Such alerts can, for example, encouraging a user to check-in with the therapeutic application 112. Check-in can include, for example, a user opening up the therapeutic application 112 and interacting with the therapeutic application 112.

In some implementations, interacting with the therapeutic application 112 can begin with the conversation engine 131 communicating 131b with the user of the user device 110 via the conversation engine API 116. The communication 131b can include, for example, a general inquiry, by the conversation engine 131, about the user's mood. In some implementations, the communication 131b is transmitted by the server 130 across the network 120 to the user device 110. The user device 110 can obtain the communication 131b and generate output data that presents the communication 131b to the user of the user device 110. The output data can be presented to the user of the user device 110 in a number of different ways. For example, the output data can be include displaying a message, from the conversation engine 131, to the user in an interface in a messaging application format that shows dialogue from the conversation engine 131 on one side of the messaging application interface and dialogue from the user, when received, on the other side of the messaging application interface. However, the present disclosure need not be so limited and, instead, in some implementations, the communications 131a can be presented to the user via audio output from a speaker. In some implementations, for example, the conversation engine 131 can transmit a communication 131b that asks the user “how are you feeling today?”

The user of the user device 110 can input a response 131c to the communication 131b into the therapeutic application 112. The conversation engine API 116 can transmit the response 131c to the server 130 via the network 120. The conversation engine 131 can provide the response 131c as an input to the mood evaluation engine 132. The mood evaluation engine 132 is configured to receive the response 131c from the conversation engine 131. The mood evaluation engine 132 can evaluate the response 131c and determine, based on the response 131c, a likely mood of the user of the user device 110. For example, in some implementations, the mood evaluation engine 132 can provide the response 131c as an input to a machine learning model that has been trained to process response 131c data and generate output data corresponding to a mood of the user based on processing of the response 131c data. Data corresponding to the mood of the user device 110 can include, for example, data indicating a likely mood of the user and a confidence score indicating a confidence of the machine learning model that the predicted mood is correct. In such implementations, the machine learning model can be trained on training data items corresponding to response data provided by prior patients, response data provided by patients of a clinical trial, or the like. In some implementations, such as the implementation depicted by FIG. 1, the mood evaluation engine can output a confidence score and associate the confidence score with the mood of the user of the user device 110 determined by the mood evaluation engine 132. In such implementations, the present disclosure can determine, at decisioning engine 133, whether the confidence score of the determined mood satisfies a predetermined threshold. If the determined confidence score satisfies the predetermined threshold, then the decisioning engine 133 can determine that the mood evaluation engine determined the likely mood of the user of the user device 110 with a sufficient level of accuracy and the system 100 can provide data 133a identifying a likely mood of the user of the user device 110 to the diagnosis engine 134. Diagnosis as used herein, refers to either a clinical diagnosis or a non-clinical assessment of symptoms. Alternatively, if the determined confidence score does not satisfy the predetermined threshold, then the decisioning engine 133 can determine that the mood evaluation engine did not determine the likely mood of the user of the user device 110 with a sufficient level of accuracy and the system 100 can instruct 133b the conversation engine 131 to interact with the user of the user device 110 in order to obtain additional response data from the user that can be used, by the mood evaluation engine 132, to sufficiently determine a likely mood of the user of the user device. The system 100 can continue this iterative feedback loop until the decision engine 133 determines that the mood evaluation engine 132 has sufficiently determined a likely mood of the user of the user device 110. Aspects of this process for the determining a likely mood of user of the user device 110 based on the conversation engine's 131 interaction with the user are discussed in more detail below.

In some implementations, the communication 131b from the conversation engine 131 to the user of the user device 110 can include a single natural language communication (e.g., how are you feeling today?) and the response 131c can be a single natural language response (e.g., I feel depressed). In such implementations, the conversation engine 131 can provide the single communication 131b, the single response 131c, or both, as inputs to the mood evaluation engine 132. Then, the mood evaluation engine 132 can determine (i) a likely mood of the user of the user device 110 based on the single communication 131b, the single response 131c, or both and (ii) a confidence score indicating a likelihood that the determined likely mood is sufficient. If the decisioning engine 133 determines that the determined likely mood is not sufficient, then the decisioning engine 133 can instruct the conversation engine 131 to communicate another natural language communication to the user in order to elicit another natural language response that can be provided to mood evaluation engine 132 in order to make a subsequent determination regarding a likely mood of the user of the user device 110 and an associated confidence score. This process can be performed, iteratively, until the decisioning engine 133 determines that the likely mood determined by the mood evaluation engine is sufficient to provide the determined likely mood as an input to the diagnosis engine 134. However, the present disclosure is not so limited and other implementations are discussed below.

In some implementations, for example, the system 100 is not limited a single natural language communication from the conversation engine 131 and a single natural language response from the user of the user device 110. Instead, for example, there can be an iterative cycle of natural language communications from the conversation engine 131 and natural language responses from the user of the user device 110, with the iterative cycle of natural language communications and natural language responses forming a conversation thread between the conversation engine 131 and the user of the user device 110. In such implementations, the entire conversation thread, or a portion thereof, may be provided as an input to the mood evaluation engine 132. Then, the mood evaluation engine 132 can determine (i) a likely mood of the user of the user device 110 based on the conversation thread and (ii) a confidence score indicating a likelihood that the determined likely mood is sufficient. In some examples, the mood evaluation engine may use natural language processing, including machine learning as described above, to determine the likely mood. For example, in some implementations, the mood evaluation engine 132 can provide conversation thread, or any portion thereof, as an input to a machine learning model that is trained to process a conversation thread, or portion thereof, to generate output data corresponding to a mood of the user based on process of the conversation thread, or portion thereof. Data corresponding to the mood of the user device 110 can include, for example, data indicating a likely mood of the user and a confidence score indicating a confidence of the machine learning model that the predicted mood is correct. In such implementations, the machine learning model can be trained on training data items corresponding to a conversation thread, or portion thereof, communicated between a conversation engine and one or more prior patients such as, e.g., patients of a clinical trial. If the decisioning engine 133 determines that the determined likely mood is not sufficient, then the decisioning engine can instruct the conversation engine 131 to continue to communicate with the user of the user device 110 using one or more natural language communications in order to elicit one or more other natural language responses that can be provided, alone or in combination with all or a portion of the current conversation thread, to the mood evaluation engine 132 in order to make a subsequent determination regarding a likely mood of the user of the user device 110 and an associated confidence score. This process can be performed, iteratively, until the decisioning engine 133 determines that the likely mood determined by the mood evaluation engine is sufficient to provide the determined likely mood as an input to the diagnosis engine 134. However, the present disclosure is not so limited and other implementations are discussed below.

In some implementations, for example, the system 100 can interact with the user of the user device 110 transmitting a communication that includes a plurality of pre-programmed button options. Pre-programmed buttons can be represented in a graphical user interface of the therapeutic application 112 using a selectable graphical icon. Each pre-programmed button can correspond to an attribute of an emotional state (e.g., a mood). Emotional states that can be associated with a pre-programmed button can include, e.g., anxious, really anxious, sick, really sick, angry, really angry, happy, really happy, content, sad, really sad, depressed, really depressed, okay, tired, really tired, or the like. The user's response to a communication from the conversation engine 131 that includes pre-programmed buttons can include a selection of one or more of the pre-preprogrammed buttons. In such implementations, response data indicating the pre-programmed buttons selected by the user of the user device 110, may be provided as an input to the mood evaluation engine 132. Then, the mood evaluation engine 132 can determine (i) a likely mood of the user of the user device 110 based on the response data indicating the pre-programmed buttons selected by the user of the user device 110 and (ii) a confidence score indicating a likelihood that the determined likely mood is sufficient. For example, in some implementations, the mood evaluation engine 132 can provide response data indicating the pre-programmed buttons selected by the user of the user device 110 as an input to a machine learning model that has been trained to process data indicating pre-programmed buttons selected by the user of a user device and generate output data corresponding to a mood of the user of the user device 110. Data corresponding to the mood of the user device 110 can include, for example, data indicating a likely mood of the user and a confidence score indicating a confidence of the machine learning model that the predicted mood is correct. In such implementations, the machine learning model can be trained on training data items corresponding to pre-programmed buttons selected by prior patients (e.g., patients of a clinical trial). If the decisioning engine 133 determines that the determined likely mood is not sufficient, then the decisioning engine 133 can instruct the conversation engine 131 to communicate one or more additional pre-programmed buttons to the user in order to elicit additional response data corresponding to one or more selections of the additional pre-programmed buttons that can be provided to mood evaluation engine 132 in order to make a subsequent determination regarding a likely mood of the user of the user device 110 and an associated confidence score. This process can be performed, iteratively, until the decisioning engine 133 determines that the likely mood determined by the mood evaluation engine is sufficient to provide the determined likely mood as an input to the diagnosis engine 134. However, the present disclosure is not so limited and other implementations are discussed below.

In some implementations, the conversation engine 131 can communicate with the user of the user device 110 using a combination of natural language and pre-programmed buttons. In such implementations, for example, the communication 131b from the conversation engine 131 can begin with an initial natural language communication (e.g., how are you feeling today?) and the response 131c can be a single natural language response (e.g., I feel depressed). In such implementations, the conversation engine 131 can provide the single communication 131b, the single response 131c, or both, as inputs to the mood evaluation engine 132. Then, the mood evaluation engine 132 can determine (i) a likely mood of the user of the user device 110 based on the single communication 131b, the single response 131c, or both and (ii) a confidence score indicating a likelihood that the determined likely mood is sufficient. For this implementation, assume that the decisioning engine 133 determines that the determined likely mood is not sufficient, then the decisioning engine 133 instructs the conversation engine 131 to further communicate with the user of the user device 110 to elicit additional response data that can be provided to the mood evaluation engine 132 in order to make a subsequent determination regarding a likely mood of the user of the user device 110 and an associated confidence score. However, in this implementation, the decisioning engine 133 instructs the conversation engine 131 to provide a communication to the user of the user device 110 that includes one or more pre-programmed buttons that each correspond to an emotional state to elicit response data indicating a selection of one or more of the pre-programmed buttons. The conversation engine 131 can receive the response data indicating a selection of one or more of the pre-programmed buttons and provide the response data indicating a selection of one or more of the pre-programmed buttons as an input to the mood evaluation engine 132. Then, the mood evaluation engine 132 can determine (i) a likely mood of the user of the user device 110 based on the response data indicating the pre-programmed buttons selected by the user of the user device 110 and (ii) a confidence score indicating a likelihood that the determined likely mood is sufficient. If the decisioning engine 133 determines that the determined likely mood is not sufficient, then the decisioning engine can instruct the conversation engine 131 to communicate one or more additional natural language communications and/or one or more additional pre-programmed buttons to the user in order to elicit additional response data comprising additional natural language responses from the user of the user device 110 and/or response data indicating one or more selections of the additional pre-programmed buttons that can be provided to mood evaluation engine 132 in order to make a subsequent determination regarding a likely mood of the user of the user device 110 and an associated confidence score. This process can be performed, iteratively, until the decisioning engine 133 determines that the likely mood determined by the mood evaluation engine is sufficient to provide the determined likely mood as an input to the diagnosis engine 134. However, the present disclosure is not so limited and other implementations are discussed below.

The above examples describe conversational techniques that can be employed by the conversational engine 131 in order to elicit information from the user of the user device 110 that is indicative of the user's mood. These conversational techniques are intended to be exemplary in nature and not limiting. In addition, it should be understood that these conversational techniques should not be viewed in isolation and features of each of these conversational techniques can be used across implementations.

Importantly, in some implementations, these conversational techniques can be iterative in nature, thus enabling the conversational engine 131 to establish a therapeutic alliance with the user of the user device 110 that functions to improve adherence to the therapeutic application 112. Once the decisioning 133 has determined, using these conversational techniques, a likely mood of the user of the user device 110, the decisioning engine 133 can provide data 133a indicating a likely mood of the user as an input to the diagnosis engine 134.

The diagnosis engine 135 can determine, based on data 133a indicating a likely mood of the user of the user device 110, digital therapeutic content from one or more of database 140, 141, 142 that can be administered to the user of the user device 110 in order to treat one or more symptoms of anxiety, depression, stress or burnout. In some implementations, the one or more databases can include a cognitive behavioral therapy (CBT) Tools database 140, an interpersonal psychotherapy (IPT) tools database 141, and anxiety or depression specific database 142, each of which will be described in more detail below.

The CBT tools database 140 can include broad-based cognitive behavioral therapy (CBT) lessons that are configured to treat symptoms that are not necessarily specific to a particular mental health condition (e.g., anxiety, depression, stress, or burnout). In some implementations, for example, CBT lessons stored in the CBT Tools database include broad-based content designed in a manner that, when consumed by an individual exhibiting one or more symptoms of a mental health condition, causes a therapeutic effect on the individual that treats the one or more symptoms of the mental health condition. Though the CBT Tool content stored in CBT tools database can treat the symptoms of one or more mental health conditions, the CBT Tool content is not specifically designed for a particular mental health condition. This means that, by way of example, a therapeutic content module stored in the CBT Tool database 140 may be equally effective for treating an individual exhibiting symptoms of anxiety or depression as it would be for a patient exhibiting symptoms of another mental health condition. This may be, e.g., because the therapeutic content may be designed, e.g., to just cheer the individual up without having any explicit content that discusses or describes aspects of anxiety or depression.

In some implementations, these CBT Tools are designed to intervene on cognitions by, e.g., challenging and/or changing automatic thinking patterns and behaviors through behavioral activation. In some implementations, one or more of the respective CBT Tools can be configured to include a psychological assessment, psychoeducation about CBT and aspects of one or more mental health conditions, mood monitoring, practice with CBT tools (e.g., thought challenging), practice sessions, or any combination thereof.

In some implementations, CBT Tools are configured to cause a change in thinking patterns and behaviors through behavioral activation. Behavioral activation encourages participants to engage in positive, pleasurable behaviors in order to manage and/or improve an individual's mood. Behavioral activation CBT Tools can include therapeutic content that triggers the release of chemicals such as, e.g., dopamine, in the brain of an individual viewing the therapeutic content. Such CBT tools that activate such biological responses in an individual impact the individual's mood, with the released chemicals causing an improvement in the individual's mood. Behavioral activation also addresses behavioral avoidance and other barriers to engaging in such behaviors using CBT Tools to teach problem solving techniques and alternative coping strategies, helping people have more regulated and higher functioning lives. Accordingly, CBT Tools utilizing behavioral activation techniques can therapeutically treat an individual exhibiting one or more symptoms of anxiety or depression, which may cause an individual to feel unhappy or insecure, by triggering release of chemicals within an individual's body that counteract those feelings on unhappiness or insecurity by causing the individual to feel happiness, confidence, and positivity.

In some implementations, one or more of the CBT Tools can be configured to interface between the conversation engine 131 and the conversation engine API 116 of the therapeutic application 112, thus enabling interactivity between the user of the user device 110 and the conversation engine 131 within the context of the CBT Tool. In such implementations, the CBT Tool can comprise natural language conversation back and forth between the user and the conversation engine 131, with the conversation engine 131 storing user responses in a memory location that is associated with the user's user of the CBT Tool. In such instances, the conversation engine 131 can draw on the user's previous responses during prior therapeutic treatments using the CBT Tool during subsequent therapeutic treatments using the CBT Tool, as the conversation engine 131 can recall prior interactions with the user using the CBT Tool based on the stored responses.

The Interpersonal Psychotherapy (IPT) Tool database 141 can include psychotherapy lessons that provide intervention that can be used to treat anxiety or depression. In particular, IPT tools work to alleviate clinical symptomatology by helping an individual improve interpersonal functioning. IPT has been proven to benefit mental health conditions such as depression based in attachment and interpersonal theory postulates that depressive symptomatology has biopsychosocial roots, and as a result, consequences of interpersonal distress fall into one of a plurality of predefined problem areas including role disputes, role transitions, grief, and interpersonal deficits. Like the content in the CBT Tool database 140, though the IPT Tools in IPT Tool database 141 can be used to treat symptoms of anxiety or depression, each IPT Tool need not necessarily be specific to a particular mental health condition, and can provide emotional support and help live better without bothersome symptoms of depression, anxiety, stress and burnout.

The anxiety or depression (A/D) specific database 142 can include psychotherapy lessons that are specifically tailored to address symptoms of anxiety or depression, stress and mood. In contrast to the CBT Tools database and IPT Tools database, which each include content in the form of CTB Tools or IPT Tools that are designed independent of a particular mental health condition, A/D database 142 includes content that are designed specifically to treat one or more particular symptoms of anxiety or depression. For example, the A/D specific lessons database 142 may include therapeutic content that has been specifically designed to treat symptoms related to anxiety or depression using lessons designed to improve a person's mood and have explicit discussions and/or teachings related to the topics of anxiety or depression.

The example of FIG. 1 describes the database 142 as being an anxiety or depression (A/D) specific database 142, the present disclosure is not so limited. Instead, for example, the database 142 should be understood to be a database storing condition-specific psychotherapy lessons. The example of FIG. 1 describes treatment anxiety or depression. Thus, the condition-specific psychotherapy database 142 is an anxiety or depression (A/D) database. However, the system 100 is equally applicable to treat conditions such as stress or burnout. In such implementations, the condition specific database 142 would be a stress or burnout (S/B) database that stores condition-specific psychotherapy lessons that are designed to treat symptoms of stress or burnout.

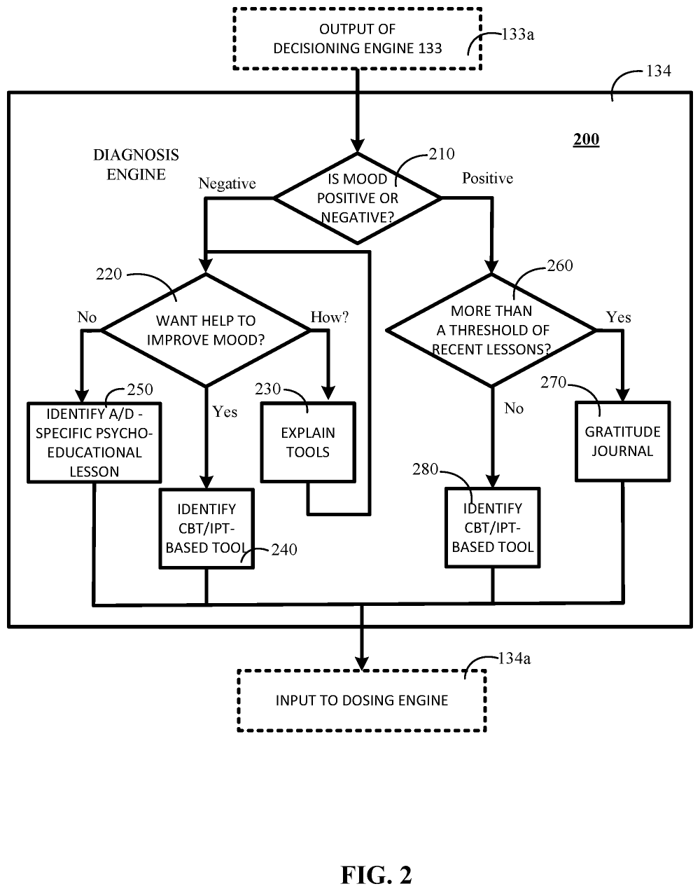

The execution of the system 100 is further discussed with reference to FIG. 2, which is a flowchart of an example of a process 200 that can be executed by a diagnosis engine 131, in accordance with one aspect of the present disclosure. As shown in FIG. 2 (and FIG. 1), execution of the system 100 can continue with the decisioning engine 133 providing data 133a indicating a likely mood of the user of the user device 110 to the diagnosis engine 134. The diagnosis engine 134 can use a decisioning engine 210 to determine whether the data 133a indicating a likely mood of the user indicates a positive mood or a negative mood. If the decisioning engine 210 determines that the data 133a indicates that the mood of the user of the user device 110 is negative, then the execution of the process 200 can continue to decisioning engine 220.

Here, the decisioning engine 220 can instruct the conversation engine 131 to ask the user of the user device 110 if the user wants help improving the user's mood. In one case, the user of the user device 110 may submit a response that asks the conversation engine 132 how the therapeutic application 112 can help to improve the user's mood. In such instances, the decisioning engine 220 of the dosing engine 134 can instruct the conversation engine 132 to explain 230 the therapeutic tools that can be provided by the therapeutic application 112. Such an explanation may include, e.g., a description and/or tutorial regarding anxiety or depression specific psychoeducational lessons, a description and/or tutorial regarding cognitive behavioral therapy (CBT) based tools, a description and/or tutorial regarding interpersonal theory (IPT) based tools, or any combination thereof. In some implementations, the conversation engine 131 can provide links, e.g., to tutorial content that describes each of the therapeutic tools of anxiety or depression specific psychoeducational lessons, CBT based tools, and/or IPT based tools. In such instances, the user has the discretion to view the tutorials for one or more of the therapeutic tools or emotional support.

During the execution of the process 200 performed by the diagnosis engine 134 within the context of the system of FIG. 1, a second case can occur during operation of the decisioning engine 220. For example, in some instances, a user may respond to the prompting for help with his/her mood by indicating that, yes, the user would like help improving the user's mood. In such instances, the decisioning engine 220 of the diagnosis engine 134 can identify 240 one or more CBT and/or IPT based tools stored in the CBT Tools DB 140 or IPT Tools DB 141. In some implementations, the identification of the CBT Tools and/or IPT Tools by the diagnosis engine 134 can be based on the diagnosis engine's 134 analysis of the response data received from the user during the user's interaction with the conversation engine 131. As described above, response data can include (i) a single natural language response, (ii) multiple natural language responses, (iii) data indicating selections of one or more pre-programmed buttons corresponding to different emotional states (e.g., moods), (iv) or a combination thereof.

During the execution of the process 200 performed by the diagnosis engine within the context of the system of FIG. 1, a third case can occur during operation of the decisioning engine 220. For example, in some instances, a user may respond to the prompting for help with his/her mood by indicating that, no, the user would not like help improving the user's mood. In such instances, the decisioning engine 220 of the diagnosis engine 134 can identify 250 one or more anxiety or depression (A/D) specific psychoeducational lessons stored in the anxiety or depression (A/D) specific lesson database 142. In some implementations, the identification of the anxiety or depression (A/D) specific psychoeducational lessons by the diagnosis engine 134 can be based on the diagnosis engine's 134 analysis of the response data received from the user during the user's interaction with the conversation engine 131. As described above, response data can include (i) a single natural language response, (ii) multiple natural language responses, (iii) data indicating selections of one or more pre-programmed buttons corresponding to different emotional states (e.g., moods), (iv) or a combination thereof. In other implementations, the diagnosis engine may already have existing information such as, e.g., a user profile, indicating one or more symptoms of anxiety or depression a user is exhibiting and identify anxiety or depression specific psychoeducational lessons for the user based on the existing information.

The above example describes a scenario where the diagnosis engine 134 is being used to diagnosis treatment for anxiety or depression. Thus, in the example of FIG. 2, when the user's mood is determined at decisioning engine 210 to be negative and the decision engine 220 determines that the user wants help improving the user's mood, then the diagnosis engine 250 identify anxiety or depression (A/D) specific psychotherapy lessons. However, the present disclosure is not so limited. Instead, the diagnosis engine at 250 can identify condition-specific treatment (whereas treatment at stage 240 or 280 is not condition specific). Accordingly, in implementations where the system 100 is used to treat stress or burnout, the diagnosis engine 200 can identify at 250 stress or burnout (S/B) specific psychoeducational lessons from database 142.

The above operations of the diagnosis engine 131 are performed by executing the operations described by the process 200 when the decisioning engine 210 of the diagnosis engine 134 determines that the likely mood of the user of the user device 110 is negative. Alternatively, however, the decisioning engine 210 can initiate performance of different operations responsive to a determination, by the decisioning engine 210, that the data 133a indicates that the user of user device 110 has a mood that is likely positive.

Here, the decisioning engine 210 can determine that the data 133a indicates that the likely mood of the user is positive. Based on a determination that the likely mood of the user of user device 110 is positive, the decisioning engine 210 can instruct the decisioning engine 260 to evaluate the number of recent lessons completed by the user of the user device 110. In a first case, the decisioning engine 260 can determine that the user of the user device 110 has recently consumed (e.g., watched therapeutic content, listened to therapeutic content, read therapeutic content, or a combination thereof) more than a threshold number of recent therapeutic lessons. Based on a determination that the user of the user device 110 has recently completed more than a threshold number of therapeutic lessons, the decisioning engine 260 of the diagnosis engine can cause the therapeutic application 112 to display a gratitude journal in the display of the therapeutic application 112. The gratitude journal provides, e.g., the user of the user device 110 a mechanism to log the user's positive thoughts in a journal, the action of which may reinforce the user's positive feelings. Alternatively, if the decisioning engine 260 determines that the user of the user device has not recently completed more than a threshold number of therapeutic lessons, the decisioning engine 260 of the diagnosis engine 200 can identify 280 one or more CBT and/or IPT based tools stored in the CBT Tools DB 140 or IPT Tools DB 141. For purposes of this disclosure, “recently” can include a threshold period of time that precedes a point in time when the decisioning engine 260 of the diagnosis engine 134 evaluates the number of therapeutic lessons the user has completed. The threshold period of time can include a predetermined number of hours, days, weeks, or the like. In some implementations, the identification of the CBT Tools and/or IPT Tools by the diagnosis engine 134 can be based on the diagnosis engine's analysis of the response data received from the user during the user's interaction with the conversation engine 131. As described above, response data can include (i) a single natural language response, (ii) multiple natural language responses, (iii) data indicating selections of one or more pre-programmed buttons corresponding to different emotional states (e.g., moods), (iv) or a combination thereof.

The output data 134a of the diagnosis engine 134 is data that identifies one or more therapeutic content items that are to be administered to the user. For purposes of this disclosure, data that identifies one or more therapeutic content items that are to be administered to the user of the user device 110 can include data that references the one or more therapeutic content items (e.g., one or more URLs), data that indicates the one or more therapeutic content items (e.g., data that the user device 110 can use to identify or unlock therapeutic content), or one or more therapeutic content media files.

Data that references one or more therapeutic content items can be processed by the user device 110 or selected by the user of the user device 110 in order to access the therapeutic content items that are to be administered to the user of the user device 110. Such reference data may link to therapeutic content items that are stored locally on the user device 110 or remotely from a user device 110. Data stored remotely from the user device 110 may be stored in one or more databases such as, e.g., database 140, 141, 142. In some implementations, data that references one or more therapeutic content items can include, for example, a URL.

Data that indicates one or more therapeutic content items can be processed by a user device 110 in order to instruct the user device as to a particular set of one or more therapeutic content items that are to be administered to a user of the user device 110. In some implementations, data that indicates one or more therapeutic content items can include, for example, a content identifier for a therapeutic content item or group of one or more therapeutic content items. In such implementations, the therapeutic application 112 on the user device 110 may have access to a library of therapeutic content items stored locally on the user device 110 or remotely from the user device 110 that is accessible by the user device 110. Then, in such implementations, the user device 110 may use the data indicating the one or more therapeutic content items to identify one or more particular therapeutic content items from the library of therapeutic content items and administer the one or more identified particular therapeutic content items to the user of the user device 110.

In some implementations, data that indicates one or more therapeutic content items can include an authorization key, a security key, private key, a license, or the like for one or more therapeutic content items. In such implementations, the therapeutic application 112 on the user device 110 may have access to a library of therapeutic content items that are stored locally on user device 110 or remotely from the user device 110. However, in this implementation, the one or more therapeutic content items accessible by the therapeutic application 112 of the user device 110 may have restricted access. Restricted access can include content that is access controlled, encrypted, or otherwise protected. In such implementations, receipt of a data indicating one or more particular therapeutic content items such as an authorization key, security key, private key, license, or the like gives the therapeutic application 112 of the user device 110 the authority to access and unlock the particular therapeutic content items that correspond to the received indicating data. Such an implementation provides the benefit of prohibiting a user of a user device 110 from accessing therapeutic content items that were not identified by the diagnosis engine 200 to treat the user of the user device 110 (or otherwise prescribed for the user).

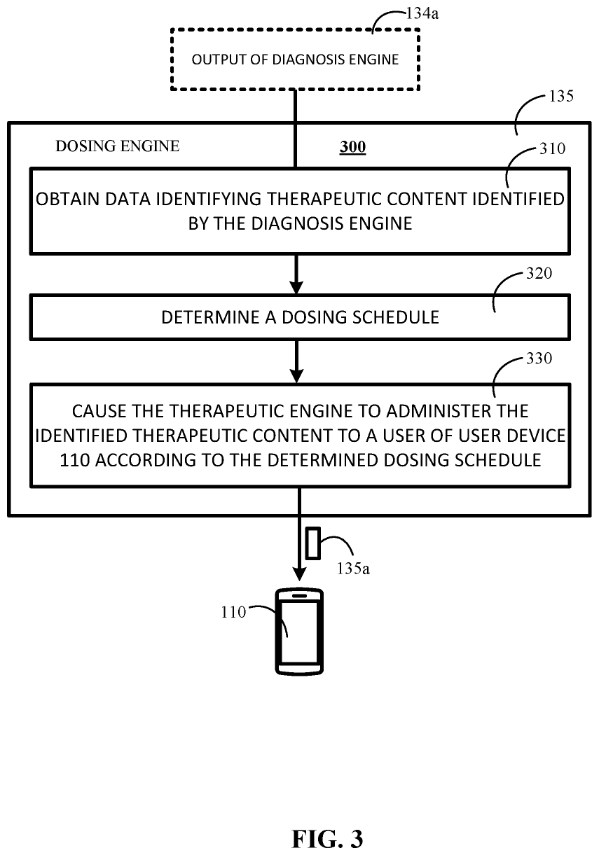

The diagnosis engine 134 can provide this output data 134a to the dosing engine 134a. The operations performed by the dosing engine 134a within the context of the system of FIG. 1 are described with reference to the process of FIG. 3. With reference to FIG. 3, the dosing engine 135 can obtain 310 output data 134a of the diagnosis engine 134 that identifies one or more therapeutic content items that are to be administered to a user of the user device 110. The one or more therapeutic content items may include one or more CBT Tools, one or more IPT tools, or one or more anxiety or depression (A/D) specific lessons.

The dosing engine can determine 320 a dosing schedule for the user. The dosing schedule can indicate a frequency of treatment, a duration of treatment, or both. A frequency of treatment indicates a number of treatments per specific time period (e.g., hour, day, week, or month, or the like). A duration of treatment indicates how many time periods the treatment is to be administered (e.g., 10 days, 2 weeks, 8 weeks, 2 to 8 weeks, at least 8 weeks, 3 months, 1-3 months, or the like). Thus, by way of example, a dosing schedule determined by the dosing engine can be for the user to consume the prescribed content for at least 3 times per week for a duration of two to eight weeks. However, other frequencies of treatment and durations of treatment can be prescribed by the dosing engine 135 based on a conversation thread between the user of the user device 110 and the conversation engine 131, the user's mood as determined by an analysis of a conversation thread between the user of the user device and the conversation engine 131, or the like. In some implementations, a persistent symptom mitigation engine 136 may provide information about a user's treatment to the diagnosis engine 135 and request that the dosing engine 135 determine if the user's dosing schedule is to be adjusted. For example, based on feedback information obtained by the persistent symptom mitigation engine 136, the dosing engine 136 can increase a frequency of treatment, decrease a frequency of treatment, increase a duration of treatment, decrease a duration of treatment, or any combination thereof. Modifications to an existing dosing schedule can be made, by the dosing engine 135, based on, e.g., symptoms of a mental health condition detected by the persistent symptom mitigation engine, a number of recent lessons completed, trending emotional behavior responsive to treatment determined based on user feedback, or the like.

In some implementations, the dosing engine 135 can determine that a longer, initial duration of treatment is beneficial to a user of the user device 110. An example of a longer duration treatment prescribed by the dosing engine 135 may include, for example, a dosing schedule of 3 times a week for at least 8 weeks. Though the examples used herein describe a frequency of 3 times per time period for a duration of treatment, the present disclosure is not so limited. Instead, the dosing engine 135 may determine any frequency of treatment per time period. The therapeutic application 112 can be configured to monitor the user's adherence to the specified dosing schedule and intervene to the extent the user strays from the dosing schedule. For example, upon determination that the user has strayed from the dosing schedule, the conversation engine 131 can trigger an accountability conversation with the user to get the user back on schedule, explain a paradoxical injunction (e.g., a paradoxical cost benefit analysis), or the like.

The dosing engine can cause 330 the therapeutic engine 118 of the therapeutic application 112 to administer the therapeutic content to the user of the user device 110 according to the determined dosing schedule. This can include, for example, transmitting the therapeutic regimen data 135a to the user device 110. The therapeutic regimen data 135a can include (i) the obtained therapeutic content, data that references the therapeutic content, and/or data that indicates the therapeutic content, and (ii) the dosing schedule determined at stage 320. The data 135a can also include instructions that cause the triggering engine 114 to alert the user of the user device 110 as to the receipt of the therapeutic regimen data 135a in order to trigger initiation of the administration of treatment. Alternatively, in other implementations, the dosing engine 135, or other component of system 100 such as the conversation engine 131, can transmit a separate notification (e.g., a push notification) to the user device 110 that causes the triggering engine 114 to alert the user of the user device 110 to the receipt of the therapeutic regimen data 135a. In this alternative implementation, the dosing schedule 135a can, but is not required to, include data that alerts the user of the user device 135a regarding the receipt of the therapeutic regimen data 135a.

Alternatively, in the case of the therapeutic content being the gratitude journal, the dosing engine 135 can obtain data indicating how to access the gratitude journal, determine a dosing schedule indicating a frequency with which the user of the user device 110 is to write in the gratitude journal, and then cause the treatment engine to administer the treatment that includes instructions, to the user, to add an entry to the gratitude journal in accordance with the frequency specified by the dosing schedule. The therapeutic application 112 can be configured to monitor the user's adherence to the specified dosing schedule and intervene to the extent the user strays from the dosing schedule. For example, upon determination by the therapeutic application 112 that the user has strayed from the dosing schedule, the conversation engine 131 can be informed of the user straying and then trigger an accountability conversation with the user to get the user back on schedule, explain a paradoxical injunction (e.g., a paradoxical cost benefit analysis), or the like.

The user device 110 can receive the therapeutic regimen data 135a from server 130. Based on the receipt of the therapeutic regimen data 135a or an accompanying notification, the triggering engine 114 can alert the user of the user device 110 regarding the receipt of the therapeutic regimen data 135a and prompt the user of the user device 110 to check in for administration of the therapeutic treatment. In some implementations, the user can check-in responsive to the receipt of the notification indicating that the therapeutic regimen has been received and begin treatment. In other implementations, the therapeutic application 112 may provide the user with an option to delay start of the therapeutic treatment in a manner that is consistent with the therapeutic regimen data 135. This means that, by way of example, if the therapeutic regimen 135a requires that the user consume one or more therapeutic content items for 2 times per week for a certain duration of weeks, then the therapeutic application will only permit the user of the user device up to 5 days of delay so that the user still has 2 days remaining in the week to complete the treatment for that week. In some implementations, if the user does not adhere to the dosing regimen 135a, the therapeutic application 113 can be configured to intervene in a manner that causes the user to get back on the dosing regimen. For example, in some implementations, upon determination by the therapeutic application 112 that the user has strayed from the dosing schedule of a dosing regimen, the conversation engine 131 can be informed of the user straying and then trigger an accountability conversation with the user to get the user back on schedule, explain a paradoxical injunction (e.g., a paradoxical cost benefit analysis), or the like.

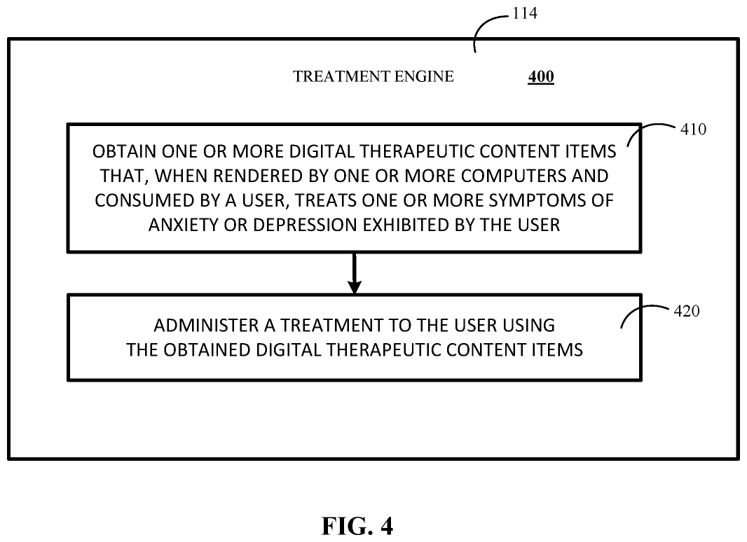

The therapeutic application 112 can use the therapeutic engine 118 to administer a therapeutic treatment to the user of the user device 110 using the one or more therapeutic content items after receipt of the therapeutic regimen 13a. The process for using the therapeutic engine 118 to administer a therapeutic treatment to the user of the user device is described with reference to FIG. 4. With reference to FIG. 4, the therapeutic engine 118 can begin execution of the process 400 by obtaining (410) one or more digital therapeutic content items identified by the therapeutic regime data 135a. This digital therapeutic content includes, for example, one or more one or more CBT Tools, one or more IPT tools, or one or more anxiety or depression specific lessons. These digital therapeutic content items, when rendered by the user device 110 and consumed by a user, treats one or more symptoms of the user's anxiety or depression.

The therapeutic engine 118 can obtain 410 the one or more digital therapeutic content items in a number of different ways based on the manner in which the therapeutic regimen data 135a identifies the one or more digital therapeutic content items. For example, if the therapeutic regimen data 135a includes a reference such as a URL to one or more digital therapeutic content items, the therapeutic engine 118 can obtain the URL, access the URL, and obtain the one or more digital therapeutic content items referenced by the URL. Alternatively, if the therapeutic regimen includes data indicating one or more digital therapeutic content items, then the therapeutic engine 118 can use the data indicating the one or more digital therapeutic content items to access one or more digital therapeutic content items using the data from the therapeutic regimen data that indicates the one or more digital therapeutic content items. Data indicating the therapeutic content item can include, for example, a content identifier, authorization key, security key, private key, license, or the like.

The therapeutic engine 118 can administer 420 a treatment to the user using the obtained one or more digital therapeutic content items. In some implementations, administering the treatment can include rendering, by the user device 110, the one or more obtained digital therapeutic content items on a graphical user interface of the user device 110 when the user is viewing the graphical user interface, a predetermined number of times a week for a duration of two to eight weeks. In some implementations, the predetermined number of times is three times per week.

In some implementations, the therapeutic application 112 can employ various techniques to determine whether a user is viewing the graphical user interface of the user device 110 when the one or more digital therapeutic content items are being rendered. For example, in some implementations, the therapeutic application 112 can periodically prompt or randomly prompt the user to select the display of a graphical element that was silently displayed on the graphical user interface of the user device 110. If the user fails to select the displayed graphical element, the therapeutic application 112 can determine that the user is not viewing the graphical user interface of the user device 110 and perform one or more operations (e.g., alerting user with an audible alert, alerting user with a haptic feedback alert, or the like) in order to direct the user's attention back to the digital therapeutic content item being rendered on the graphical user interface of the user device 110.

In some implementations, the therapeutic application 112 can obtain and interpret sensor data of the user device 110 in order to determine whether the user of the user device 110 is viewing the graphical user interface of the user device 110. For example, the therapeutic application 112 can determined based on, for example, sensor data from a gyroscope, accelerometer, or both, the orientation of the user device 110 and whether the current orientation of the user device 110 is an orientation in which the user is likely to hold the user device 110 when looking at the graphical user interface of the user device 110. Another example can include the user device 110 periodically or randomly capturing images from a user-facing camera of the user device 110. In such instances, the therapeutic application 112 can perform image analysis on the captured images to determine if the user is currently looking at the graphical user interface of the user device 110. If the therapeutic application 112 determines that the user is not viewing the graphical user interface of the user device 110, the therapeutic application can perform one or more operations (e.g., alerting user with an audible alert, alerting user with a haptic feedback alert, or the like) in order to direct the user's attention back to the digital therapeutic content item being rendered on the graphical user interface of the user device 110.

Other, optional, features may be employed by the system 100. For example, in some implementations, the system 100 can be modified to include optional features such as the crisis mitigation. In such alternative implementations, the mood evaluation engine 132 can be configured to evaluate response data from the user device 110 to detect whether a predetermined level of depression, anxiety, or emotional crisis is present in the user's responses. Response data can include, for example, a single natural language response, multiple natural language responses, a conversation thread, data indicating selection of one or more pre-programmed buttons, or any combination thereof. Based on a determination, by the mood evaluation engine 132, that a predetermined level of depression, anxiety, or emotional crisis has been detected in the user's response data, the mood evaluation engine 132 can instruct the conversation engine 131 to perform an intervention with the user. In some implementations, the intervention can include natural language communication from the conversation engine 131 that is designed to improve the user's mood. Alternatively, or in addition, the intervention can include the conversation engine providing links to content that, when consumed by the user, causes the user's mood to improve. Alternatively, or in addition, the intervention can include the conversation engine 131 alerting an emergency contact, police department, or fire department in an effort to cause the emergency contact to intervene.

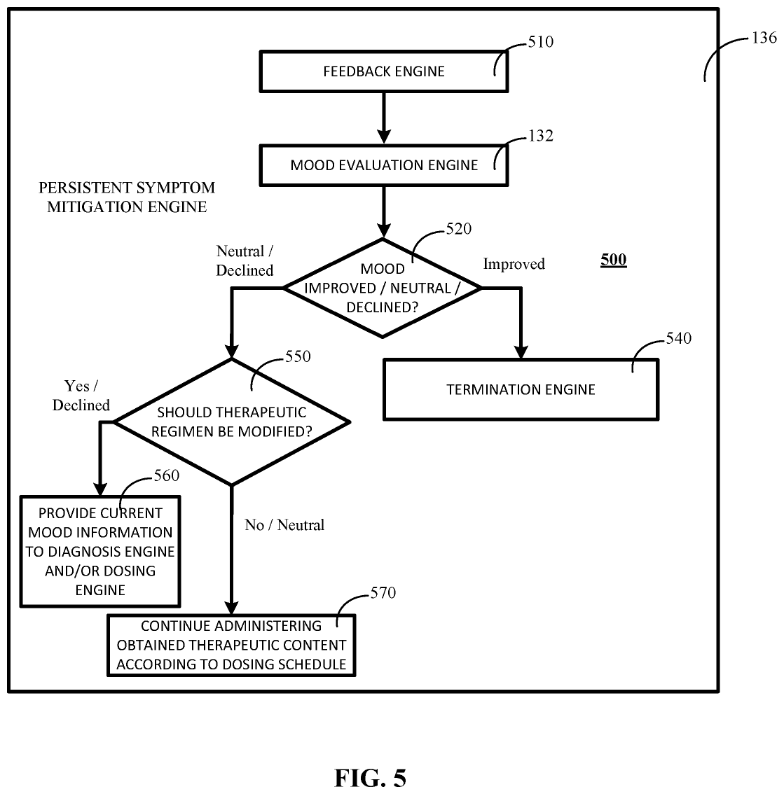

FIG. 5 is a flowchart of an example of a process 500 that can be executed by a persistent symptom mitigation engine 136, in accordance with one aspect of the present disclosure. Execution of the process 500 by the persistent symptom mitigation engine 136 utilizes the mood evaluation engine 132 of FIG. 1.

The persistent symptom mitigation engine 136 can use a feedback engine 510 to obtain feedback from a user of the user device 110 regarding therapeutic treatment received. In some implementations, the feedback engine 510 can transmit a message 136a to the user of the user device 110 that can be accessed via the therapeutic application 112. This message can include, for example, a survey of one or more questions in the form of an email message, text message, or the like. In such implementations, the feedback engine 510 can await receipt of the user's responses to the survey of one or more questions before continuing execution of the process 500. Alternatively, in some implementations, the feedback engine 510 can instruct the conversation engine 131 to interact with the user of the user device 110 in order to elicit feedback regarding the therapeutic treatment from the user of the user device 110. Then, the conversation engine 131 can provide all, or a portion of, the conversation thread to the feedback engine 510 for processing in the remainder of process 500.

The feedback engine 136 can receive feedback data 131d and provide the feedback data as an input to the mood evaluation engine 132. The feedback data 131d can include a response to the survey of one or more questions from the feedback engine 510. Alternatively, or in addition, the feedback data 136a can include all, or a portion of, a conversation thread between the user of the user device and the conversation engine 131. The mood evaluation engine can process the feedback data 131d to determine a current mood of the user of the user device 110, a trending direction of the user's mood, or a combination thereof. The mood evaluation engine 132 can generate output data indicating a current mood of the user of the user device 110, a trending direction of the user's mood, or a combination thereof. The mood evaluation engine 132 can provide the generated output data as an input to the decisioning engine 520.

The decisioning engine 520 can determine whether the user's mood has improved, declined, or stayed neutral. If the decisioning engine 520 determines that the user's mood has improved by a threshold amount, then the decisioning engine 520 can instruct the termination engine 540 to terminate the remainder of the user's treatment specified by the therapeutic regimen data. Alternatively, if the decisioning engine 520 determines that the user's mood is either neutral or has declined, then the decisioning engine 520 can continue execution of the process 500 by instructing the decisioning engine 550 to determine whether the user's therapeutic regimen should be modified.

The decisioning engine 550 can determine whether the user's therapeutic treatment regimen should be modified. If the decisioning engine 550 determines that the user's mood has declined, then the decisioning engine can provide 560 current mood information to the diagnosis engine 134 or the dosing engine 135 in order to have the user's therapeutic treatment modified. In implementations, for example, where decisioning engine 550 provides 560 information to the diagnosis engine 134, the diagnosis engine 134 can identify therapeutic content based on the current mood information and/or content of feedback data 131d and then provide the identified therapeutic content to the dosing engine 135. Alternatively, the decisioning engine 550 can determine that user's mood is neutral. In such instances, the decisioning engine 550 can determine that the system 100 should continue 570 administering the previously obtained therapeutic content according to the dosing schedule established by the therapeutic regimen data.

Alternatively, or in addition, in implementations, for example, where the decisioning engine 550 provides 560 current mood information to the dosing engine 135, the dosing engine 135 can, based on feedback information obtained by the persistent symptom mitigation engine 136, increase a frequency of treatment, decrease a frequency of treatment, increase a duration of treatment, decrease a duration of treatment, or any combination thereof. Modifications to an existing dosing schedule can be made, by the dosing engine 135, based on, e.g., symptoms of a mental health condition detected by the persistent symptom mitigation engine, a number of recent lessons completed, trending emotional behavior responsive to treatment determined based on user feedback, or the like.

FIG. 6 is a diagram of an example of a user device 610 that is configured to generate, dose, and administer a digital therapeutic to treat one or more symptoms related to anxiety or depression. Regarding the user device 610, the user device 610 is similar to the user device 110, except the user device 510 is configured to execute the entirety of system 100 within the user device. Specifically, this means that in addition to the triggering engine 114, the conversation engine API, and the therapeutic engine 118, the user device 510 also includes the conversation engine 131, the mood evaluation engine 132, the decisioning engine 133, the diagnosis engine 134, the dosing engine 135, and the persistent symptom mitigation engine 136. Each of these respective engines performs the same operations described with reference to system 100, but in the implementation of FIG. 6, are installed on the user device 610 and, thus, reside locally on the user device 610. Likewise, the database 140, 141, 142 are also depicted as residing locally on the user device 610. And, in some implementations, the databases 140, 141, and 142 may reside locally on the user device 610. However, for purposes of the present disclosure, there is no requirement that the database 140, 141, 412 reside locally on the user device 610 and, instead, can be stored remotely and accessed by the user device 610 via one or more networks.

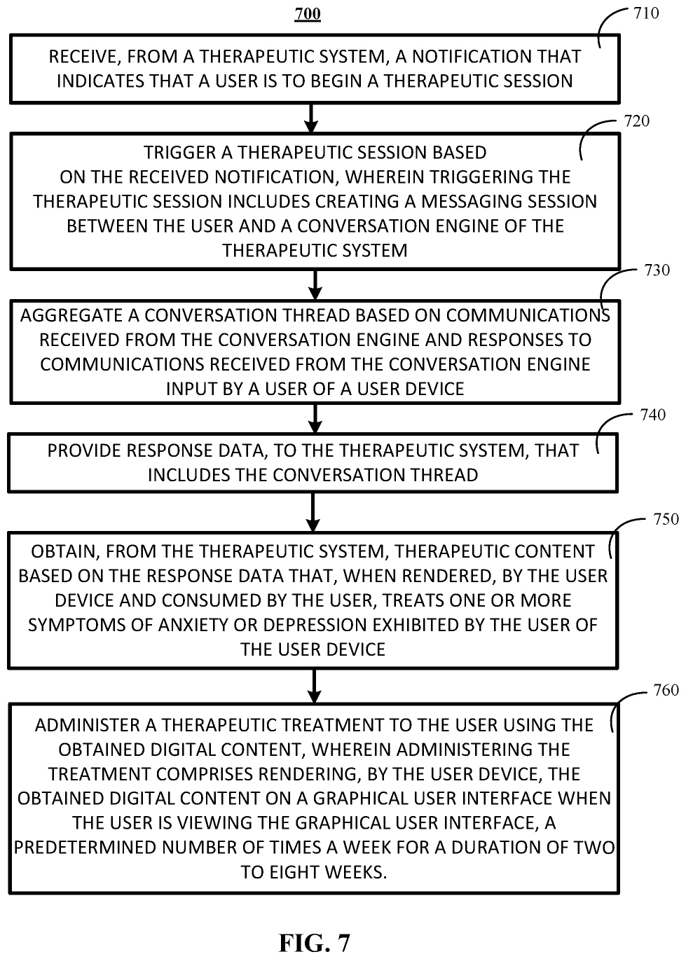

FIG. 7 is flowchart of an example of a process 700 for administering a digital therapeutic to treat anxiety or depression, in accordance with one aspect of the present disclosure. In some implementations, anxiety or depression can include postpartum depression. For convenience, the method will be described as being performed by a user device such as, e.g., the user device 110 of FIG. 1.

Execution of the process 700 can begins with a user device receiving, from a therapeutic system, a notification that indicates that a user is to begin a therapeutic session (710).

The user device can continue execution of the process 700 by triggering a therapeutic session based on the received notification, where triggering the therapeutic session includes creating of a messaging session between the user and a conversation engine of the therapeutic system (720).

The user device can continue execution of the process 700 by aggregating a conversation thread based on communications received from the conversation engine and responses to communications received from the conversation engine input by a user of the user device (730). In some implementations, aggregating the conversation thread can include collecting one or more communications or responses between the conversation engine and the user of the user device.

The user device can continue execution of the process 700 by providing response data, to the therapeutic system, that includes the conversation thread (740).

The user device can continue execution of the process 700 by obtaining, from the therapeutic system, therapeutic content based on the response data that, when rendered, by the user device and consumed by a user, treats one or more symptoms of anxiety or depression exhibited by the user (750).

The user device can continue execution of the process 700 by administering a therapeutic treatment to the user using the obtained digital content. In some implementations, administering the treatment comprises rendering, by the user device, the obtained digital content on a graphical user interface when the user is viewing the graphical user interface, a predetermined number of times a week for a duration of two to eight weeks (760).

In some implementations, the conversation thread can include at least one communication from the conversation engine and at least one communication from the user that is a response to the communication from the conversation engine.

In some implementations, the conversation thread can include (i) a single natural language communication from the conversation engine and (ii) a single natural language response from the user.

In some implementations, the conversation thread can include (i) multiple natural language communications from the conversation engine and (ii) multiple natural language responses from the user.

In some implementations, the conversation thread comprises (i) a plurality of pre-programmed graphical elements, with each of the pre-programmed graphical elements corresponding to an emotional state of the user and (ii) response data indicating selection of one or more pre-programmed graphical elements by the user.

In some implementations, the user device can continue execution of the process 700 by receiving, from the therapeutic system, a request for feedback following completion of a therapeutic session, providing, feedback data entered by the user to the therapeutic system, and receiving instructions to terminate the administered treatment prior to the completion of the full duration of the treatment prescribed by the therapeutic system.

In some implementations, obtaining, by the user device and from the therapeutic system, therapeutic content based on the response data that, when rendered, by the user device and consumed by a user, treats one or more symptoms of anxiety or depression exhibited by the user as described by process 700 can include obtaining, by the user device, one or more interpersonal psychotherapy (IPT) tools and one or more cognitive behavioral therapy (CTB) tools based on the response data.

In some implementations, administering a therapeutic treatment, by the user device, to the user using the obtained digital content, as described by process 700 can include rendering, by the user device, one or more interpersonal psychotherapy (IPT) tools and one or more cognitive behavioral therapy (CBT) tools in the user interface of the user device a predetermined number of times a week for a duration of two to eight weeks.

In some implementations, administering a therapeutic treatment, by the user device, to the user using the obtained digital content described by process 700 can include using the user device to deliver interactive cognitive behavioral therapy (CBT) that comprises natural language interaction with a conversation engine. In such implementations, natural language interaction can include (i) one or more natural language communications from the conversation engine to the user of the user device and (ii) one or more natural language responses from the user of the user device to the conversation engine.

In some implementations, administering a therapeutic treatment, by the user device, to the user using the obtained digital content described by process 700 can include using the user device to deliver an interactive therapy that comprises natural language interaction with a conversation engine. In such implementations, the natural language interaction can include (i) one or more natural language communications from the conversation engine to the user of the user device and (ii) one or more natural language responses from the user of the user device to the conversation engine.

In some implementations, the predetermined number of times a week of process 700 comprises 3 to 5 times per week.

In some implementations, the predetermined number of times a week of process 700 comprises daily use during the week.

In some implementations, the predetermined duration of process 700 is two to eight weeks.

In some implementations, the predetermined duration of process 700 is at least 8 weeks.

In some implementations, the method for treating anxiety or depression of process 700 comprises a method for treating clinical levels of anxiety or depression symptoms.

In some implementations, the therapeutic content is administered for at least 5 minutes during each of the predetermined number of times per week.

In some implementations, the therapeutic content of process 700 is administered at least 10 minutes during each of the predetermined number of times per week.

In some implementations, the predetermined number of times a week for a predetermined duration of process 700 comprises at least 4 weeks of an 8-week time period.

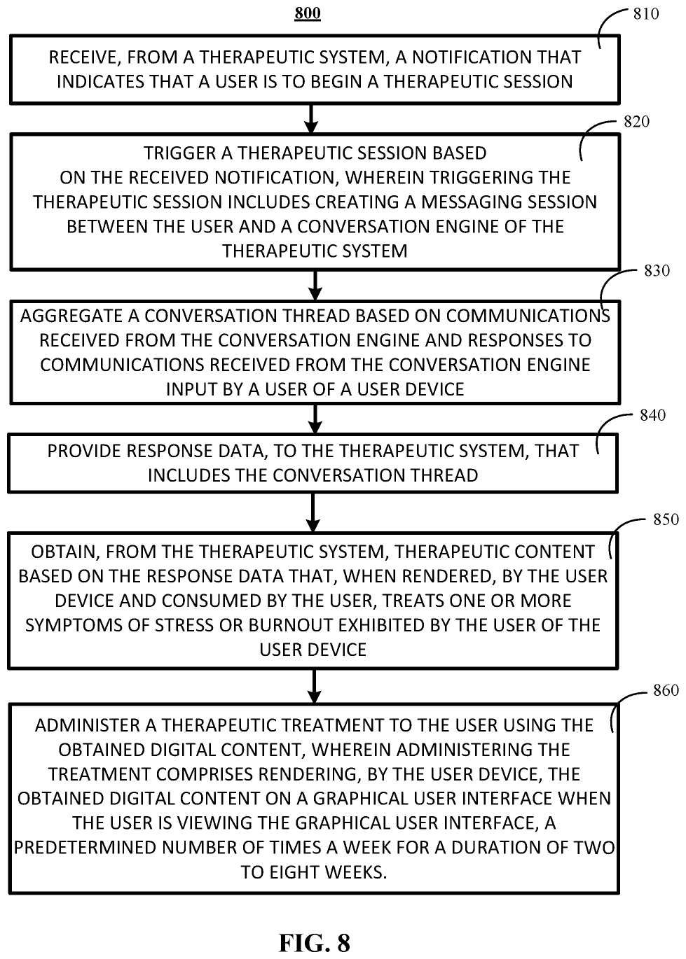

FIG. 8 is flowchart of an example of a process 800 for administering a digital therapeutic to treat stress or burnout, in accordance with one aspect of the present disclosure. For convenience, the method will be described as being performed by a user device such as, e.g., the user device 110 of FIG. 1.

Execution of the process 800 can begins with a user device receiving, from a therapeutic system, a notification that indicates that a user is to begin a therapeutic session (810).

The user device can continue execution of the process 800 by triggering a therapeutic session based on the received notification, where triggering the therapeutic session includes creating of a messaging session between the user and a conversation engine of the therapeutic system (820).

The user device can continue execution of the process 800 by aggregating a conversation thread based on communications received from the conversation engine and responses to communications received from the conversation engine input by a user of the user device (830). In some implementations, aggregating the conversation thread can include collecting one or more communications or responses between the conversation engine and the user of the user device.

The user device can continue execution of the process 800 by providing response data, to the therapeutic system, that includes the conversation thread (840).

The user device can continue execution of the process 800 by obtaining, from the therapeutic system, therapeutic content based on the response data that, when rendered, by the user device and consumed by a user, treats one or more symptoms of stress or burnout exhibited by the user (850).

The user device can continue execution of the process 800 by administering a therapeutic treatment to the user using the obtained digital content. In some implementations, administering the treatment comprises rendering, by the user device, the obtained digital content on a graphical user interface when the user is viewing the graphical user interface, a predetermined number of times a week for a duration of two to eight weeks (860).

In some implementations, the conversation thread of process 800 can include at least one communication from the conversation engine and at least one communication from the user that is a response to the communication from the conversation engine.

In some implementations, the conversation thread of process 800 can include (i) a single natural language communication from the conversation engine and (ii) a single natural language response from the user.

In some implementations, the conversation thread of process 800 can include (i) multiple natural language communications from the conversation engine and (ii) multiple natural language responses from the user.

In some implementations, the conversation thread of process 800 comprises (i) a plurality of pre-programmed graphical elements, with each of the pre-programmed graphical elements corresponding to an emotional state of the user and (ii) response data indicating selection of one or more pre-programmed graphical elements by the user.

In some implementations, the user device can continue execution of the process 800 by receiving, from the therapeutic system, a request for feedback following completion of a therapeutic session, providing, feedback data entered by the user to the therapeutic system, and receiving instructions to terminate the administered treatment prior to the completion of the full duration of the treatment prescribed by the therapeutic system.

In some implementations, obtaining, by the user device and from the therapeutic system, therapeutic content based on the response data that, when rendered, by the user device and consumed by a user, treats one or more symptoms of stress or burnout exhibited by the user as described by process 800 can include obtaining, by the user device, one or more interpersonal psychotherapy (IPT) tools and one or more cognitive behavioral therapy (CTB) tools based on the response data.

In some implementations, administering a therapeutic treatment, by the user device, to the user using the obtained digital content, as described by process 800 can include rendering, by the user device, one or more interpersonal psychotherapy (IPT) tools and one or more cognitive behavioral therapy (CBT) tools in the user interface of the user device a predetermined number of times a week for a duration of two to eight weeks.

In some implementations, administering a therapeutic treatment, by the user device, to the user using the obtained digital content as described by process 800 can include using the user device to deliver interactive cognitive behavioral therapy (CBT) that comprises natural language interaction with a conversation engine. In such implementations, natural language interaction can include (i) one or more natural language communications from the conversation engine to the user of the user device and (ii) one or more natural language responses from the user of the user device to the conversation engine.

In some implementations, administering a therapeutic treatment, by the user device, to the user using the obtained digital content as described by process 800 can include using the user device to deliver an interactive therapy that comprises natural language interaction with a conversation engine. In such implementations, the natural language interaction can include (i) one or more natural language communications from the conversation engine to the user of the user device and (ii) one or more natural language responses from the user of the user device to the conversation engine.

In some implementations, the predetermined number of times a week of process 800 can include 3 to 5 times per week.

In some implementations, the predetermined number of times a week of process 800 can include daily use during the week.

In some implementations, the predetermined duration of process 800 is two to eight weeks.

In some implementations, the predetermined duration of process 800 is at least 8 weeks.

In some implementations, the method for treating stress or burnout described by process 800 can include a method for treating clinically elevated levels of stress or burnout.

In some implementations, the therapeutic content of process 800 is administered for at least 5 minutes during each of the predetermined number of times per week.

In some implementations, the therapeutic content of process 800 is administered at least 10 minutes during each of the predetermined number of times per week.

In some implementations, the predetermined number of times a week for a predetermined duration of process 800 comprises at least 4 weeks of an 8-week time period.

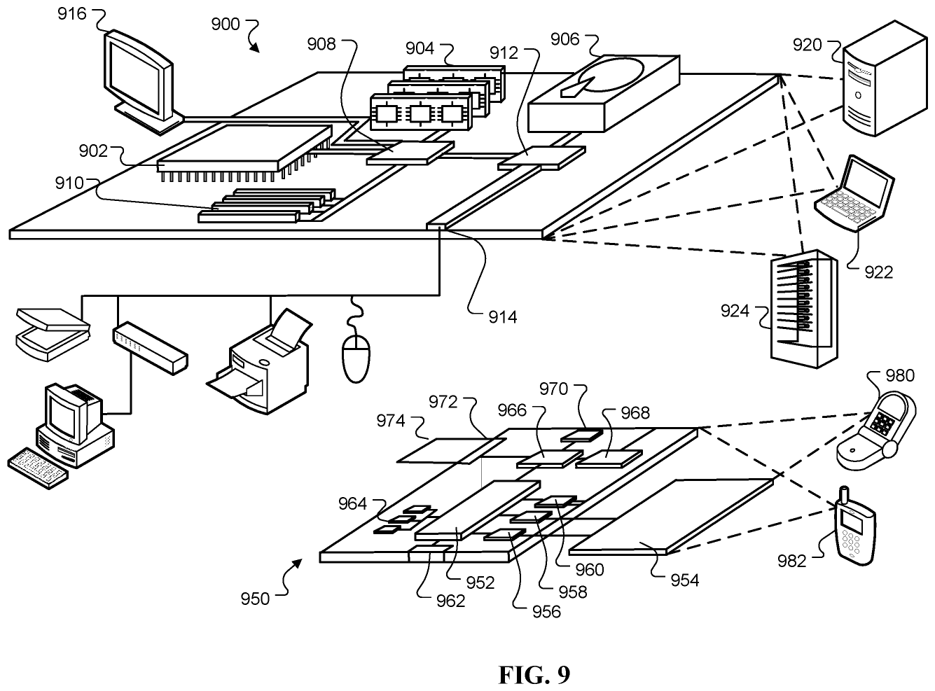

FIG. 9 is a block diagram of system components that can be used to implement a system for generating, dosing, and administering a digital therapeutic to one or more symptoms related to a mental health condition.

Computing device 900 is intended to represent various forms of digital computers, such as laptops, desktops, workstations, personal digital assistants, servers, blade servers, mainframes, and other appropriate computers. Computing device 950 is intended to represent various forms of mobile devices, such as personal digital assistants, cellular telephones, smartphones, and other similar computing devices. Additionally, computing device 900 or 950 can include Universal Serial Bus (USB) flash drives. The USB flash drives can store operating systems and other applications. The USB flash drives can include input/output components, such as a wireless transmitter or USB connector that can be inserted into a USB port of another computing device. The components shown here, their connections and relationships, and their functions, are meant to be exemplary only, and are not meant to limit implementations of the inventions described and/or claimed in this document.

Computing device 900 includes a processor 902, memory 904, a storage device 906, a high-speed interface 908 connecting to memory 904 and high-speed expansion ports 910, and a low-speed interface 912 connecting to low-speed bus 914 and storage device 906. Each of the components 902, 904, 906, 908, 910, and 912, are interconnected using various busses, and can be mounted on a common motherboard or in other manners as appropriate. The processor 902 can process instructions for execution within the computing device 900, including instructions stored in the memory 904 or on the storage device 906 to display graphical information for a GUI on an external input/output device, such as display 916 coupled to high-speed interface 908. In other implementations, multiple processors and/or multiple buses can be used, as appropriate, along with multiple memories and types of memory. Also, multiple computing devices 900 can be connected, with each device providing portions of the necessary operations, e.g., as a server bank, a group of blade servers, or a multi-processor system.

The memory 904 stores information within the computing device 900. In one implementation, the memory 904 is a volatile memory unit or units. In another implementation, the memory 904 is a non-volatile memory unit or units. The memory 904 can also be another form of computer-readable medium, such as a magnetic or optical disk.

The storage device 906 is capable of providing mass storage for the computing device 900. In one implementation, the storage device 906 can be or contain a computer-readable medium, such as a floppy disk device, a hard disk device, an optical disk device, or a tape device, a flash memory or other similar solid state memory device, or an array of devices, including devices in a storage area network or other configurations. A computer program product can be tangibly embodied in an information carrier. The computer program product can also contain instructions that, when executed, perform one or more methods, such as those described above. The information carrier is a computer- or machine-readable medium, such as the memory 904, the storage device 906, or memory on processor 902.

The high-speed controller 908 manages bandwidth-intensive operations for the computing device 900, while the low-speed controller 912 manages lower bandwidth intensive operations. Such allocation of functions is exemplary only. In one implementation, the high-speed controller 908 is coupled to memory 904, display 916, e.g., through a graphics processor or accelerator, and to high-speed expansion ports 910, which can accept various expansion cards (not shown). In the implementation, low-speed controller 912 is coupled to storage device 906 and low-speed expansion port 914. The low-speed expansion port, which can include various communication ports, e.g., USB, Bluetooth, Ethernet, wireless Ethernet can be coupled to one or more input/output devices, such as a keyboard, a pointing device, microphone/speaker pair, a scanner, or a networking device such as a switch or router, e.g., through a network adapter. The computing device 900 can be implemented in a number of different forms, as shown in the figure. For example, it can be implemented as a standard server 920, or multiple times in a group of such servers. It can also be implemented as part of a rack server system 924. In addition, it can be implemented in a personal computer such as a laptop computer 922. Alternatively, components from computing device 900 can be combined with other components in a mobile device (not shown), such as device 950. Each of such devices can contain one or more of computing device 900, 950, and an entire system can be made up of multiple computing devices 900, 950 communicating with each other.

The computing device 900 can be implemented in a number of different forms, as shown in the figure. For example, it can be implemented as a standard server 920, or multiple times in a group of such servers. It can also be implemented as part of a rack server system 924. In addition, it can be implemented in a personal computer such as a laptop computer 922. Alternatively, components from computing device 900 can be combined with other components in a mobile device (not shown), such as device 950. Each of such devices can contain one or more of computing device 900, 950, and an entire system can be made up of multiple computing devices 900, 950 communicating with each other

Computing device 950 includes a processor 952, memory 964, and an input/output device such as a display 954, a communication interface 966, and a transceiver 968, among other components. The device 950 can also be provided with a storage device, such as a micro-drive or other device, to provide additional storage. Each of the components 950, 952, 964, 954, 966, and 968, are interconnected using various buses, and several of the components can be mounted on a common motherboard or in other manners as appropriate.

The processor 952 can execute instructions within the computing device 950, including instructions stored in the memory 964. The processor can be implemented as a chipset of chips that include separate and multiple analog and digital processors. Additionally, the processor can be implemented using any of a number of architectures. For example, the processor 910 can be a CISC (Complex Instruction Set Computers) processor, a RISC (Reduced Instruction Set Computer) processor, or a MISC (Minimal Instruction Set Computer) processor. The processor can provide, for example, for coordination of the other components of the device 950, such as control of user interfaces, applications run by device 950, and wireless communication by device 950.

Processor 952 can communicate with a user through control interface 958 and display interface 956 coupled to a display 954. The display 954 can be, for example, a TFT (Thin-Film-Transistor Liquid Crystal Display) display or an OLED (Organic Light Emitting Diode) display, or other appropriate display technology. The display interface 956 can comprise appropriate circuitry for driving the display 954 to present graphical and other information to a user. The control interface 958 can receive commands from a user and convert them for submission to the processor 952. In addition, an external interface 962 can be provide in communication with processor 952, so as to enable near area communication of device 950 with other devices. External interface 962 can provide, for example, for wired communication in some implementations, or for wireless communication in other implementations, and multiple interfaces can also be used.

The memory 964 stores information within the computing device 950. The memory 964 can be implemented as one or more of a computer-readable medium or media, a volatile memory unit or units, or a non-volatile memory unit or units. Expansion memory 974 can also be provided and connected to device 950 through expansion interface 972, which can include, for example, a SIMM (Single In Line Memory Module) card interface. Such expansion memory 974 can provide extra storage space for device 950, or can also store applications or other information for device 950. Specifically, expansion memory 974 can include instructions to carry out or supplement the processes described above, and can include secure information also. Thus, for example, expansion memory 974 can be provide as a security module for device 950, and can be programmed with instructions that permit secure use of device 950. In addition, secure applications can be provided via the SIMM cards, along with additional information, such as placing identifying information on the SIMM card in a non-hackable manner.

The memory can include, for example, flash memory and/or NVRAM memory, as discussed below. In one implementation, a computer program product is tangibly embodied in an information carrier. The computer program product contains instructions that, when executed, perform one or more methods, such as those described above. The information carrier is a computer- or machine-readable medium, such as the memory 964, expansion memory 974, or memory on processor 952 that can be received, for example, over transceiver 968 or external interface 962.

Device 950 can communicate wirelessly through communication interface 966, which can include digital signal processing circuitry where necessary. Communication interface 966 can provide for communications under various modes or protocols, such as GSM voice calls, SMS, EMS, or MMS messaging, CDMA, TDMA, PDC, WCDMA, CDMA2000, or GPRS, among others. Such communication can occur, for example, through radio-frequency transceiver 968. In addition, short-range communication can occur, such as using a Bluetooth, Wi-Fi, or other such transceiver (not shown). In addition, GPS (Global Positioning System) receiver module 970 can provide additional navigation- and location-related wireless data to device 950, which can be used as appropriate by applications running on device 950.

Device 950 can also communicate audibly using audio codec 960, which can receive spoken information from a user and convert it to usable digital information. Audio codec 960 can likewise generate audible sound for a user, such as through a speaker, e.g., in a handset of device 950. Such sound can include sound from voice telephone calls, can include recorded sound, e.g., voice messages, music files, etc. and can also include sound generated by applications operating on device 950.

The computing device 950 can be implemented in a number of different forms, as shown in the figure. For example, it can be implemented as a cellular telephone 980. It can also be implemented as part of a smartphone 982, personal digital assistant, or other similar mobile device.

Various implementations of the systems and methods described here can be realized in digital electronic circuitry, integrated circuitry, specially designed ASICs (application specific integrated circuits), computer hardware, firmware, software, and/or combinations of such implementations. These various implementations can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms “machine-readable medium” “computer-readable medium” refers to any computer program product, apparatus and/or device, e.g., magnetic discs, optical disks, memory, Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor.

To provide for interaction with a user, the systems and techniques described here can be implemented on a computer having a display device, e.g., a CRT (cathode ray tube) or LCD (liquid crystal display) monitor for displaying information to the user and a keyboard and a pointing device, e.g., a mouse or a trackball by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input.

The systems and techniques described here can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the systems and techniques described here, or any combination of such back end, middleware, or front end components. The components of the system can be interconnected by any form or medium of digital data communication, e.g., a communication network. Examples of communication networks include a local area network (“LAN”), a wide area network (“WAN”), and the Internet.

The computing system can include clients and servers. A client and server are generally remote from each other and typically interact through a communication network. The relationship of client and server arises by virtue of computer programs running on the respective computers and having a client-server relationship to each other.

Embodiments can be implemented in digital electronic circuitry, or in computer hardware, firmware, software, or in combinations thereof. Apparatus of the invention can be implemented in a computer program product tangibly embodied or stored in a machine-readable storage device for execution by a programmable processor; and method actions can be performed by a programmable processor executing a program of instructions to perform functions of the invention by operating on input data and generating output. The invention can be implemented advantageously in one or more computer programs that are executable on a programmable system including at least one programmable processor coupled to receive data and instructions from, and to transmit data and instructions to, a data storage system, at least one input device, and at least one output device. Each computer program can be implemented in a high-level procedural or object-oriented programming language, or in assembly or machine language if desired; and in any case, the language can be a compiled or interpreted language.

Suitable processors include, by way of example, both general and special purpose microprocessors. Generally, a processor will receive instructions and data from a read-only memory and/or a random-access memory. Generally, a computer will include one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including by way of example semiconductor memory devices, such as EPROM, EEPROM, and flash memory devices; magnetic disks such as internal hard disks and removable disks; magneto-optical disks; and CD_ROM disks. Any of the foregoing can be supplemented by, or incorporated in, ASICs (application-specific integrated circuits).

Other embodiments are within the scope and spirit of the description claims. Due to the nature of software, functions described above can be implemented using software, hardware, firmware, hardwiring, or combinations of any of these. Features implementing functions may also be physically located at various positions, including being distributed such that portions of functions are implemented at different physical locations.

### Examples—Anxiety or Depression

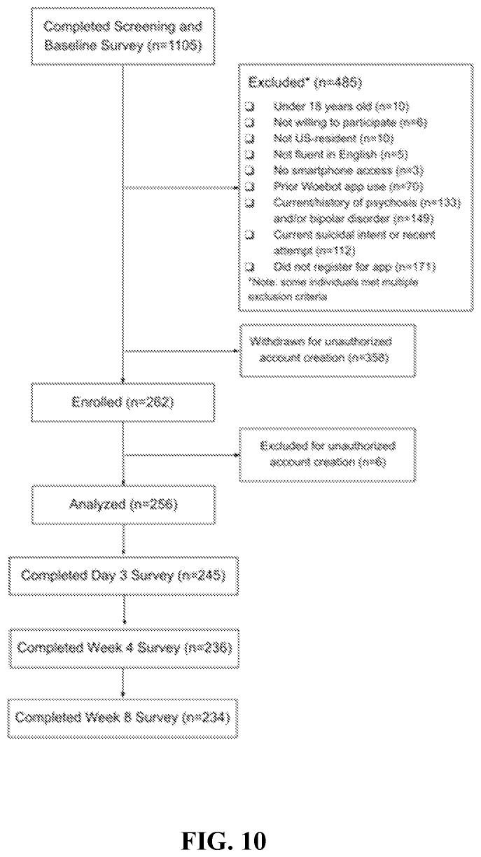

FIG. 10 is a CONSORT diagram describing participant selection and participate in a study that used the present disclosure to treat anxiety or depression. As detailed in the CONSORT of FIG. 9, 1105 potential participants were screened. Of these, 485 were excluded for not meeting eligibility criteria. After enrollment, an additional 358 participants with unauthorized accounts (e.g., duplicate registrants) were withdrawn from the study and excluded from the analyses. An additional six unauthorized registrants completed study procedures and were subsequently removed from the analytic sample, given that they were initially missed as a result of a clerical error. Thus, the final sample included in analyses consisted of 256 participants. Of these 256 participants, 245 completed the day 3 survey, 236 completed the week 4 surveys, and 234 completed the week 9 surveys.

Although Day 3 survey data were collected as part of the parent study, they were not part of the analyses included in the present report.

### Sample Characteristics

The analytic subsamples included 111 participants with elevated baseline levels of depressive symptoms and 107 participants with elevated baseline levels of anxiety symptoms at baseline as defined by a PHQ-8 score ≥10 and/or GAD-7 score ≥10, respectively. The sample size for the combined (elevated on PHQ-8 or GAD-7) groups comprised 139 participants. Some participants with elevated symptoms of both depressive and anxiety symptoms were in both analytic subsamples (n=79). Both the depressive symptom and anxiety symptom samples were predominantly female, educated, and employed, identified as non-Hispanic white and heterosexual, and had private health insurance (Table 1) with mean ages of 38 and 37 years, respectively. Among those with clinical levels of depressive symptoms, 50% had symptoms falling in the moderately severe or severe range (PHQ-8 score ≥15) and 49% had concurrent mental health treatment during the study. Among those with clinical levels of anxiety symptoms, 44% had symptoms falling in the severe range (GAD-7 score ≥15) and 53% had concurrent mental health treatment during the study.

The assessment completion rate at Week 9 was 91%. The PP population examined in sensitivity analyses comprised 177 (69%) of the 256 participants in the analytic sample after removing 67 (26%) who used WB-LIFE for at most 3 of 9 weeks and removing 22 (9%) whose end of study (EOS) PHQ-8 and GAD-7 assessments at Week 9 were fully missing (no item scores were completed for each measure); the removals were not mutually exclusive. Sixty-four percent (N=71/111) of the elevated depressive symptoms subgroup met per protocol criteria and 59% (N=63/107) of the elevated anxiety symptoms subgroup met these criteria.

TABLE 1
Baseline sociodemographic and clinical characteristics,
adherence, and satisfaction among participants with clinical
levels of baseline depressive or anxiety symptoms.

| | |
|---|---|
| Participants | Participants |
| with PHQ-8 | with GAD-7 |
| baseline score ≥10 | baseline score ≥10 |
| N = 111 | N = 107 |
| n (%) | n (%) |

Sociodemographic Characteristic

| | | | | |
|---|---|---|---|---|
| Age in years (mean, sd) | 38 | (13.57) | 37 | (12.28) |
| Race/ethnicity | | | | |
| Non-Hispanic Black | 37 | (33%) | 32 | (30%) |
| Non-Hispanic White | 55 | (50%) | 56 | (52%) |
| Other | 19 | (17%) | 19 | (18%) |
| Sex at Birth | | | | |
| Female | 83 | (75%) | 81 | (76%) |
| Male | 28 | (25%) | 26 | (24%) |
| Sexual Orientation | | | | |
| Sexual Minority | 19 | (17%) | 19 | (18%) |
| Heterosexual | 92 | (83%) | 88 | (82%) |
| Education | | | | |
| Graduate or | 32 | (29%) | 24 | (23%) |
| Postgraduate Degree | | | | |
| College Degree | 43 | (39%) | 43 | (41%) |
| Some College or | 21 | (19%) | 22 | (21%) |
| technical school | | | | |
| High School | 14 | (13%) | 16 | (15%) |
| (Grades 9-12) | | | | |
| Age in years | 38 | (13.57) | 37 | (12.28) |
| (mean, sd) | | | | |
| Employment | | | | |
| Full Time Employed | 53 | (49%) | 57 | (54%) |
| Part Time Employed | 11 | (10%) | 9 | (9%) |
| Not Employed | 29 | (27%) | 22 | (21%) |
| Other | 15 | (14%) | 17 | (16%) |
| Marital Status | | | | |
| Divorced/Separated/ | 10 | (9%) | 10 | (10%) |
| Widowed | | | | |
| Married/Partnered/ | 50 | (47%) | 48 | (46%) |
| Cohabitating | | | | |
| Single | 47 | (44%) | 47 | (45%) |
| Health Insurance | | | | |
| Government Based | 40 | (37%) | 37 | (36%) |
| Insurance | | | | |
| Private Insurance | 46 | (43%) | 45 | (43%) |
| No Insurance/Prefer | 21 | (20%) | 22 | (21%) |
| not to answer | | | | |

Clinical Characteristics

| | | | | |
|---|---|---|---|---|
| Baseline Depressive | | | | |
| symptom severity | | | | |
| Minimal | 0 | (0%) | 7 | (7%) |
| Mild | 0 | (0%) | 21 | (20%) |
| Moderate | 55 | (50%) | 25 | (23%) |
| Moderate-severe | 30 | (27%) | 29 | (27%) |
| Severe | 26 | (23%) | 25 | (23%) |
| Age in years (mean, sd) | 38 | (13.57) | 37 | (12.28) |
| Baseline Anxiety | | | | |
| symptom severity | | | | |
| Minimal | 3 | (3%) | 0 | (0%) |
| Mild | 29 | (26%) | 0 | (0%) |
| Moderate | 37 | (33%) | 60 | (56%) |
| Severe | 42 | (38%) | 47 | (44%) |
| Concurrent Mental Health | | | | |
| Treatment* | | | | |
| Any (Concurrent treatment) | 54 | (49%) | 57 | (53%) |
| None (Woebot only) | 57 | (51%) | 50 | (47%) |

Adherence Metrics

| | | | | |
|---|---|---|---|---|
| App Utilization | | | | |
| Use of WB-LIFE in at | 74 | (67%) | 69 | (64%) |
| least 4 of 9 weeks | | | | |
| Per protocol data set | 71 | (64%) | 63 | (59%) |

GAD-7 = Generalized Anxiety DIsorder-7 item scale;
PHQ-8 = Patient Health Questionnaire-8 item scale
*Concurrent mental health treatment = any psychotherapy or psychotropic medication use at any time during the study
“Per protocol” was defined as using WB-LIFE in at least 4 of 9 study weeks and completing end of study PHQ-8 and GAD-7 assessments.

### Depressive Symptoms

In the subgroup with clinically elevated depressive symptoms, PHQ-8 scores significantly decreased over the 9-week study period (mean change −7.28, p<0.001). In bivariate regression models, the amount of symptom decline did not significantly differ by age, educational level, employment, or baseline level of anxiety symptoms but did significantly differ across several characteristics tested (Table 2). Non-Hispanic Blacks, those who were single, and those with severe levels of baseline depressive symptoms demonstrated significantly greater amounts of decline in depressive symptoms than Non-Hispanic Whites, those who were married/partnered/cohabitating, and those with moderate levels of baseline depressive symptoms, respectively. Females, participants identifying as sexual minorities, those with private or government-based health insurance, and those in concurrent mental health treatment demonstrated significantly lower amounts of decline in depressive symptoms than males, participants identifying as heterosexuals, those with no health insurance or who declined to answer the health insurance question, and those not in concurrent mental health treatment, respectively. Per protocol analyses largely confirmed the ITT findings (Supplementary Table 1).

After removing race/ethnicity, insurance, and employment from consideration because of multicollinearity, the final multiple regression model indicated significant differences in the amount of decrease in depressive symptoms at Week 9 by sexual orientation, marital status, baseline depressive symptom severity category, and concurrent mental health treatment (Table 3) in the same directions as each bivariate model described above.

TABLE 2
Unadjusted Bivariate Linear Regression Models of Characteristics
Associated with change scores from baseline to Week 9 in
depressive symptoms among those with clinically elevated
levels of baseline depressive symptoms: PHQ-8 ≥ 10.

| | PHQ-8: Week 9 Change Scores | | |
|---|---|---|---|
| Characteristics of Interest | Estimates | 95% CI | p-value |
| Use of WB-LIFE in at | 2.9 | 0.51, 5.4 | 0.018 |
| least 4 of 9 weeks | | | |
| Age | 0.03 | −0.05, 0.12 | 0.4 |
| Race/Ethnicity | | | |
| Non-Hispanic White | Reference Level | | |
| Non-Hispanic Black | −6.0 | −8.3, −3.7 | <0.001 |
| Other | 0.02 | −2.8, 2.9 | >0.9 |
| Sex at Birth | | | |
| Male | Reference Level | | |
| Female | 2.7 | 0.09, 5.3 | 0.042 |
| Sexual Orientation: | | | |
| Heterosexual | Reference Level | | |
| Sexual Minority | 5.6 | 2.6, 9.6 | <0.001 |
| Education | | | |
| High School | Reference Level | | |
| College Degree | −1.9 | −5.6, 1.8 | 0.3 |
| Graduate or | −0.68 | −4.4, 3.1 | 0.7 |
| postgraduate degree | | | |
| Some college or | 1.1 | −3.1, 5.3 | 0.6 |
| technical school | | | |
| Employment | | | |
| Full Time | Reference Level | | |
| Not Employed | 2.4 | −0.36, 5.2 | 0.087 |
| Other | 3.1 | −0.49, 6.8 | 0.089 |
| Part Time Employed | 3.6 | −0.32, 7.4 | 0.071 |
| Marital Status | | | |
| Married/Partnered/Cohabiting | Reference Level | | |
| Divorced/Separated/Widowed | −2.2 | −6.2, 1.7 | 0.3 |
| Single | −4.1 | −6.6, −1.7 | <0.001 |
| Health Insurance: | | | |
| No Insurance/Prefer | Reference Level | | |
| not to answer | | | |
| Government based | 6.9 | 4.2, 9.7 | <0.001 |
| Insurance | | | |
| Private Insurance | 7.5 | 4.8, 10 | <0.001 |
| BL Depressive | | | |
| Symptom Severity | | | |
| Moderate | Reference Level | | |
| Moderate-Severe | −1.1 | −3.2, 1.0 | 0.3 |
| Severe | −9.3 | −12, −7.0 | <0.001 |
| BL Anxiety | | | |
| Symptom Severity | | | |
| Minimal | Reference Level | | |
| Mild | −1.6 | −9.5, 6.2 | 0.7 |
| Moderate | −2.1 | −9.9, 5.7 | 0.6 |
| Severe | −7.1 | −15, 0.71 | 0.074 |
| Concurrent Mental | 3.2 | 0.94, 5.4 | 0.006 |
| Health Treatment* | | | |

BL = baseline;
PHQ-8 = Patient Health Questionnaire-8 item scale
*Concurrent mental health treatment = any psychotherapy or psychotropic medication use at any time during the study

TABLE 3
Linear Regression Model (Multiple Regression) of change
scores from baseline to Week 9 in depressive symptoms
among those with clinically elevated levels of baseline
depressive symptoms: PHQ-8 ≥ 10.

| | PHQ-8: Week 9 Change Scores | | |
|---|---|---|---|
| Predictors | Estimates | 95% CI | p-value |
| (Intercept) | −2.46 | −10.16-5.24 | 0.527 |
| Used the app in at | 0.2 | −1.75-2.15 | 0.84 |
| least 4 of 9 weeks | | | |
| Age | 0.05 | −0.03-0.14 | 0.229 |
| Sex at Birth | | | |
| Male | Reference Level | | |
| Female | −0.96 | −3.24-1.32 | 0.405 |
| Sexual Orientation | | | |
| Heterosexual | Reference Level | | |
| Sexual Minority | 5.71 | 2.88-9.54 | <0.001 |
| Education | | | |
| High School | Reference Level | | |
| College Degree | −1.63 | −4.33-1.07 | 0.234 |
| Graduate or | −0.24 | −3.18-2.70 | 0.873 |
| postgraduate degree | | | |
| Some college or | 0.88 | −2.26-4.01 | 0.58 |
| technical school | | | |
| Marital Status | | | |
| Married/Partnered/Cohabiting | Reference Level | | |
| Divorced/Separated/Widowed | −3.86 | −7.38-−0.34 | 0.032 |
| Single | −3.75 | −5.78-−1.72 | <0.001 |
| BL Depressive | | | |
| Symptom Severity | | | |
| Moderate | Reference Level | | |
| Moderate-Severe | −0.09 | −2.44-2.25 | 0.937 |
| Severe | −5.32 | −8.16-−2.47 | <0.001 |
| BL Anxiety | | | |
| Symptom Severity | | | |
| Minimal | Reference Level | | |
| Mild | −3.97 | −10.29-2.35 | 0.215 |
| Moderate | −4.5 | −11.02-2.02 | 0.173 |
| Severe | −5.59 | −12.17-0.99 | 0.095 |
| Concurrent Mental | 2.62 | 0.80-4.43 | 0.005 |
| Health Treatment* | | | |

BL = baseline;
PHQ-8 = Patient Health Questionnaire-8 item scale
*Concurrent mental health treatment = any psychotherapy or psychotropic medication use at any time during the study
NOTE:
Final models did not consider race/ethnicity, employment, or insurance status because of multicollinearity.

### Anxiety Symptoms

Average GAD-7 scores for each subgroup of interest significantly improved over the course of the study. In the subgroup with clinically elevated anxiety symptoms at baseline, GAD-7 scores significantly decreased over the 9-week study period (mean change −7.45, p<0.001). The amount of symptom decline did not significantly differ by use of WB-LIFE at least 4 of 9 weeks, age, sex at birth, educational level, or employment, but did significantly differ across several characteristics tested (Table 4). Non-Hispanic Blacks, those who were single, those with severe levels of baseline depressive symptoms, and those with severe levels of anxiety symptoms demonstrated greater amounts of decline in anxiety symptoms than Non-Hispanic Whites, those who were married/partnered/cohabitating, those with moderate levels of baseline depressive symptoms, and those with moderate levels of baseline anxiety symptoms. Participants identifying as sexual minorities, those with private or government-based health insurance, and those in concurrent mental health treatment demonstrated lower amounts of decline in anxiety symptoms than heterosexuals, those with no health insurance or who declined to answer the health insurance question, and those without concurrent mental health treatment, respectively. Per protocol analyses largely confirmed the full sample findings, with the exception of concurrent mental health treatment reaching marginal significance (p=0.052, Supplementary Table 2).

After removing race/ethnicity, employment, and insurance status from consideration due to multicollinearity, the final multiple regression model indicated significant differences in the amount of decrease in anxiety symptoms at Week 9 by sexual orientation, marital status, baseline anxiety symptom severity category, and concurrent mental health treatment (Table 5) in the same direction as those previously described in each bivariate regression model.

TABLE 4
Unadjusted Bivariate Linear Regression Models of
change scores from baseline to Week 9 in anxiety
symptoms among those with clinically elevated levels
of baseline anxiety symptoms: GAD-7 ≥ 10

| | GAD-7: Week 9 Change Scores | | |
|---|---|---|---|
| Characteristics of Interest | Estimates | 95% CI | p-value |
| Use of WB-LIFE in at | 2.2 | −0.25, 4.7 | 0.077 |
| least 4 of 9 weeks | | | |
| Age | 0.03 | −0.07, 0.14 | 0.5 |
| Race/Ethnicity | | | |
| Non-Hispanic White | Reference Level | | |
| Non-Hispanic Black | −5.7 | −08.2, −3.2 | <0.001 |
| Other | −0.12 | −3.0, 2.8 | >0.9 |
| Sex at Birth | | | |
| Male | Reference Level | | |
| Female | 1.8 | −1.0, 4.6 | 0.2 |
| Sexual Orientation | | | |
| Heterosexual | Reference Level | | |
| Sexual Minority | 3.7 | 0.65, 6.8 | 0.018 |
| Education | | | |
| High School | Reference Level | | |
| College Degree | −1.7 | −5.3, 1.9 | 0.3 |
| Graduate or | −1.8 | −5.7, 2.1 | 0.4 |
| postgraduate degree | | | |
| Some college or | −0.28 | −4.5, 3.9 | 0.9 |
| technical school | | | |
| Employment | | | |
| Full Time | Reference Level | | |
| Not Employed | 2.1 | −1.0, 5.2 | 0.2 |
| Other | 1.2 | −2.2, 4.7 | 0.5 |
| Part Time Employed | 2.3 | −2.2, 6.8 | 0.3 |
| Marital Status | | | |
| Married/Partnered/Cohabiting | Reference Level | | |
| Divorced/Separated/Widowed | −1.5 | −5.8, 2.8 | 0.5 |
| Single | −2.9 | −5.4, −0.37 | 0.025 |
| Health Insurance | | | |
| No Insurance/Prefer | Reference Level | | |
| not to answer | | | |
| Government based Insurance | 5.1 | 2.1, 9.0 | <0.001 |
| Private Insurance | 6.8 | 3.9, 9.6 | <0.001 |
| BL Depressive | | | |
| Symptom Severity | | | |
| Minimal | Reference Level | | |
| Mild | −1.4 | −6.2, 3.4 | 0.6 |
| Moderate | −0.71 | −5.3, 3.9 | 0.8 |
| Moderate-Severe | −0.12 | −4.6, 4.4 | >0.9 |
| Severe | −7.3 | −12, −2.7 | 0.002 |
| BL Anxiety | | | |
| Symptom Severity | | | |
| Moderate | Reference Level | | |
| Severe | −4.9 | −7.1, −2.7 | <0.001 |
| Concurrent Mental | 3.2 | 0.90, 5.6 | 0.007 |
| Health Treatment* | | | |

BL = baseline;
GAD-7 = Generalized Anxiety DIsorder-7 item scale
*Concurrent mental health treatment = any psychotherapy or psychotropic medication use at any time during the study.

TABLE 5
Linear Regression Model (Multiple Regression) of
change scores from baseline to Week 9 in anxiety
symptoms among those with clinically elevated levels
of baseline anxiety symptoms: GAD-7 ≥ 10

| | GAD-7: Week 9 Change Scores | | |
|---|---|---|---|
| Predictors | Estimates | 95% CI | p-value |
| (Intercept) | −4.68 | −10.40-1.03 | 0.107 |
| Used the app in at | 1.26 | −1.06-3.59 | 0.282 |
| least 4 of 9 weeks | | | |
| Age | 0 | −0.11-0.11 | 0.975 |
| Sex at Birth: | | | |
| Male | Reference Level | | |
| Female | −1.76 | −4.63-1.10 | 0.224 |
| Sexual Orientation: | | | |
| Heterosexual | Reference Level | | |
| Sexual Minority | 4.47 | 1.20-7.74 | 0.008 |
| Education Level: | | | |
| High School | Reference Level | | |
| College Degree | −1.7 | −4.79-1.39 | 0.278 |
| Graduate or | −1.12 | −4.72-2.48 | 0.536 |
| postgraduate degree | | | |
| Some college or | −0.55 | −4.38-3.28 | 0.776 |
| technical school | | | |
| Marital Status: | | | |
| Married/Partnered/Cohabiting | Reference Level | | |
| Divorced/Separated/Widowed | −2.85 | −7.30-1.61 | 0.208 |
| Single | −4.19 | −6.69-−1.68 | 0.001 |
| BL Anxiety Symptom Severity: | | | |
| Moderate | Reference Level | | |
| Severe | −3.88 | −6.14-−1.62 | 0.001 |
| Concurrent Mental | 3.2 | 0.95-5.45 | 0.006 |
| Health Treatment* | | | |

BL = baseline;
GAD-7 = Generalized Anxiety DIsorder-7 item scale
*Concurrent mental health treatment = any psychotherapy or psychotropic medication use at any time during the study
NOTE:
Final models did not consider race/ethnicity, employment, and insurance status due to multicollinearity.

### Safety

No study participant reported an AE or serious adverse event (SAE) during the study. Of 256 participants, 151 free text inputs from 71 participants triggered WB-LIFE's potential concerning language detection algorithm. Of these, four participants confirmed being in a crisis situation (resulting in four total confirmations) and were automatically provided with resources and support as described in the Safety section of the Methods.

SUPPLEMENTAL TABLE 1
Linear Regression Model (Univariate Regressions) of change scores
from baseline to Week 9 in depressive symptoms among those
with clinically elevated levels of baseline depressive symptoms:
PHQ-8 ≥ 10 on the Per Protocol subgroup (N = 177).

| | PHQ-8: Week 9 Change Scores | | |
|---|---|---|---|
| Predictors | Estimates | 95% CI | p-value |
| Age | 0.03 | −0.05, 0.12 | 0.4 |
| Race/Ethnicity: | | | |
| Non-Hispanic White | Reference Level | | |
| Non-Hispanic Black | −4.0 | −6.7, −1.4 | 0.003 |
| Other | −0.09 | −3.3, 3.1 | >0.9 |
| Sex at Birth: | | | |
| Male | Reference Level | | |
| Female | 3.5 | 0.65, 6.4 | 0.017 |
| Sexual Orientation: | | | |
| Heterosexual | Reference Level | | |
| Sexual Minority | 5.0 | 1.8, 9.1 | 0.003 |
| Education Level: | | | |
| High School | Reference Level | | |
| College Degree | 0.64 | −3.4, 4.7 | 0.8 |
| Graduate or postgraduate degree | 1.9 | −2.3, 6.0 | 0.4 |
| Some college or technical school | 1.4 | −3.1, 5.9 | 0.5 |
| Employment: | | | |
| Full Time | Reference Level | | |
| Not Employed | −0.23 | −3.1, 2.7 | 0.9 |
| Other | −0.11 | −4.5, 4.3 | >0.9 |
| Part Time Employed | 1.8 | −2.3, 6.0 | 0.4 |
| Marital Status: | | | |
| Married/Partnered/Cohabiting | Reference Level | | |
| Divorced/Separated/Widowed | −2.5 | −6.6, 1.6 | 0.2 |
| Single | −4.0 | −6.6, −1.3 | 0.004 |
| Health Insurance: | | | |
| No Insurance/Prefer not to answer | Reference Level | | |
| Government based Insurance | 2.6 | −0.66, 5.9 | 0.11 |
| Private Insurance | 3.3 | −0.08, 6.7 | 0.055 |
| BL Depressive Symptom Severity: | | | |
| Moderate | Reference Level | | |
| Moderate-Severe | 1.0 | −3.5, 1.5 | 0.4 |
| Severe | −6.8 | −9.7, −4.0 | <0.001 |
| BL Anxiety Symptom Severity: | | | |
| Minimal | Reference Level | | |
| Mild | −1.5 | −8.9, 5.9 | 0.7 |
| Moderate | −2.6 | −10, 4.8 | 0.5 |
| Severe | −4.8 | −12, 2.6 | 0.2 |
| Concurrent Mental | 1.8 | −0.66, 4.2 | 0.2 |
| Health Treatment* | | | |

BL = baseline;
PHQ-8 = Patient Health Questionnaire-8 item scale
*Concurrent mental health treatment = any psychotherapy or psychotropic medication use at any time during the study
“Per protocol” was defined as using WB-LIFE in at least 4 of 9 study weeks and completing end of study PHQ-8 and GAD-7 assessments.

SUPPLEMENTAL TABLE 2
Linear Regression Model (Univariate Regression) of change
scores from baseline to Week 9 in anxiety symptoms among
those with clinically elevated levels of baseline anxiety
symptoms: GAD-7 ≥ 10 in the Per Protocol subgroup (N = 177).

| | GAD-7: Week 9 Change Scores | | |
|---|---|---|---|
| Predictors | Estimates | 95% CI | p-value |
| Age | 0.06 | −0.05, 0.17 | 0.3 |
| Race/Ethnicity: | | | |
| Non-Hispanic White | Reference Level | | |
| Non-Hispanic Black | −3.9 | −6.8, −1.0 | 0.009 |
| Other | −0.74 | −4.4, 2.9 | 0.7 |
| Sex at Birth: | | | |
| Male | Reference Level | | |
| Female | 3.2 | 0.01, 6.3 | 0.049 |
| Sexual Orientation: | | | |
| Heterosexual | Reference Level | | |
| Sexual Minority | 3.2 | −0.35, 6.6 | 0.077 |
| Education Level: | | | |
| High School | Reference Level | | |
| College Degree | 1.2 | −2.7, 5.1 | 0.5 |
| Graduate or | 1.6 | −2.7, 5.9 | 0.5 |
| postgraduate degree | | | |
| Some college or | 0.88 | −3.7, 5.5 | 0.7 |
| technical school | | | |
| Employment: | | | |
| Full Time | Reference Level | | |
| Not Employed | 0.36 | −2.8, 3.5 | 0.8 |
| Other | −2.6 | −6.6, 1.3 | 0.2 |
| Part Time Employed | 1.8 | −2.9, 6.4 | 0.4 |
| Marital Status: | | | |
| Married/Partnered/Cohabiting | Reference Level | | |
| Divorced/Separated/Widowed | 0 | −4.5, 4.5 | >0.9 |
| Single | −3 | −5.7, −0.21 | 0.035 |
| Health Insurance: | | | |
| No Insurance/Prefer | Reference Level | | |
| not to answer | | | |
| Government based Insurance | 2.1 | −1.3, 5.6 | 0.2 |
| Private Insurance | 4.1 | 0.59, 7.7 | 0.023 |
| BL Depressive | | | |
| Symptom Severity: | | | |
| Minimal | Reference Level | | |
| Mild | 0.58 | −4.9, 6.1 | 0.8 |
| Moderate | 0.34 | −4.9, 5.6 | 0.9 |
| Moderate Severe | 0.79 | −4.4, 5.9 | 0.8 |
| Severe | −3.2 | −8.6, 2.2 | 0.2 |
| BL Anxiety Symptom Severity: | | | |
| Moderate | Reference Level | | |
| Severe | −2.6 | −5.2, 0.04 | 0.053 |
| Concurrent Mental | 2.6 | −0.02, 5.1 | 0.052 |
| Health Treatment* | | | |

BL = baseline;
GAD-7 = Generalized Anxiety DIsorder-7 item scale
*Concurrent mental health treatment = any psychotherapy or psychotropic medication use at any time during the study
“Per protocol” was defined as using WB-LIFE in at least 4 of 9 study weeks and completing end of study PHQ-8 and GAD-7 assessments.

### Discussion

In this single-arm exploratory trial of WB-LIFE, an intervention employing an NLP-supported relational agent that converses via text-based message, the objective was to explore the magnitude of the reduction in self-reported depressive (PHQ-8) and anxiety (GAD-7) symptoms between baseline and Week 9 end of intervention as well as the associative relationships between demographic and clinical characteristics and each outcome, among those that had elevated scores on these measures at baseline. On average, study participants with elevated depressive symptoms at baseline experienced significant declines in self-reported depressive symptoms across the intervention period; study participants with elevated anxiety symptoms at baseline experienced significant declines in self-reported anxiety symptoms across the intervention period. In addition, analyses revealed significant associations between depressive and anxiety symptom changes and demographic and clinical characteristics in both bivariate and, in many cases, multiple regression models. The high assessment completion and WB-LIFE utilization rates are notable given that attrition remains a perennial problem in mobile health app studies (44), with one recent systematic review of depression DMHI app RCTs suggesting that dropout rates may range from 26% to 48%, even when participant reimbursement was included in the analysis (45). Participants in the present study were remunerated to complete assessments, but WB-LIFE's ability to establish rapidly and maintain therapeutic alliance (22) may have contributed to this high participant app utilization rate and warrants further study.

In both the clinically elevated depressive and anxiety groups, we noted significant decreases in self-reported symptoms at Week 9. The mean level of change was greater than seven points on both change in PHQ-8 among those with clinically elevated depressive symptoms at baseline as well as change in GAD-7 among those with clinically elevated anxiety symptoms at baseline, thus surpassing the typically accepted change score cutoff of five points to define a clinically significant response on these measures (46). In the absence of a control group, it is difficult to draw conclusions about the efficacy of WB-LIFE, especially given evidence that approximately one-third of untreated major depression or generalized anxiety disorder cases may spontaneously remit within 6 months (47-49).

Our study did not find significant associations between sex at birth, age, educational background, and clinical outcomes, which is consistent with prior psychotherapeutic and DMHI meta-analyses and systematic reviews (8,50-52). However, several studies of internet-based CBT (iCBT) have found better depression outcomes for females (26,53) and greater probability of non-response in anxiety disorders and depression for males (54). This could imply that DMHIs have a more uniform efficacy profile than iCBT and are therefore more similar to traditional psychotherapy across these demographic groups. Alternatively, our results may simply support the finding that age and sex at birth may be more important to the epidemiology of depression than to prognosis with an intervention (52).

The associations that were found between significant improvements in depression and anxiety symptoms and sexual orientation, concurrent mental health treatment, marital status, and baseline symptom severity suggest that these may be demographic predictors of intervention response to DMHIs employing relational agents. However, a series of purpose-designed follow-up studies to test each of these putative predictors of clinical outcome in a randomized clinical trial (RCT) design is needed before any definitive conclusions can be made. Given that LGBTQ individuals have demonstrated higher rates of depression and anxiety (55) that are further compounded by access barriers and stigma (31), DMHIs may present an unique opportunity to provide safe and accessible support to this group. LGBTQ individuals are also more likely to seek health information with digital resources than their non-LGBTQ counterparts (56) and a high proportion of people accessing digital text-based support services identify as belonging to sexual and gender minorities (57). Thus, further studies among this group should be a research priority. Finally, our study noted significant differences in the association of concurrent care involvement with changes in anxiety and depression symptoms, but this concurrent care was received independently and therefore no details on its characteristics (e.g. frequency, fidelity to psychotherapeutic treatment model, modality of delivery; name, dose, and route of psychopharmacological agents) were available. Future studies should test if this association holds in a true blended care model that tightly integrates the relational-agent-delivered DMHI with human support.

Further, historical findings regarding the link between marital status and psychotherapy outcomes have been decidedly mixed (58), but in this study we found that single or divorced/separated/widowed individuals in the elevated depression group and single individuals in the elevated anxiety group experienced more improved outcomes compared to their married/partnered/cohabiting peers. These results are somewhat consistent with a study of an online cognitive behavior intervention compared to a waiting list control that demonstrated greater improvements in depression outcomes at 4 months among separated, widowed, or divorced individuals as compared to married participants (59). Due to the importance of social support in mental health treatment (50), a more detailed assessment of factors such as relationship quality may better explain the relationship between marital status and clinical outcomes (50).

Finally, we found that greater baseline depression and anxiety symptom severity was associated with greater symptom improvements in the elevated depression and anxiety groups, respectively. It is notable that 50% of the elevated depression group reported either moderately severe or severe levels of baseline symptoms and 44% of the elevated anxiety groups reported severe symptoms. DMHIs have often been considered as interventions for mild to moderate symptoms (60), but these findings may suggest promise for populations with a broader range of severity. An alternative explanation may be that those with higher symptom severity may have more room to improve, which has been suggested in a previous DMHI meta-analysis (8).

### Examples—Stress or Burnout

As shown in the FIG. 10, 1105 potential participants were screened. Of these, 485 were excluded for not meeting eligibility criteria. Following enrollment, an additional 358 participants with unauthorized accounts (e.g., duplicate registrants) were withdrawn from the study and omitted from the analyses. An additional six unauthorized registrants completed study procedures and were removed from the analytic sample as they were initially missed due to clerical error. Thus, the final sample included 256 participants.

### Participant Characteristics

A total of 256 participants were enrolled in the study. Participants had a mean age of 39 years (SD=13.35), were largely female (72%), identified as non-Hispanic White (58%), and self-reported as heterosexual (82%). Of the total sample, 54% (n=139) reported clinically elevated levels of depression or anxiety at baseline. Significantly more female and single participants reported elevated symptomatology (78%; 43%, respectively) than not (66%; 25%, respectively). There was a significantly greater percentage of non-Hispanic black participants with elevated symptomatology (28%) than without (15%). Conversely, there were significantly less non-Hispanic White participants with elevated symptomatology (54%) than without (63%). Lastly, there was a significantly greater percentage of participants without clinical symptomatology (81%) who engaged with the app for at least 4 of 9 weeks compared to participants with clinical symptomatology (68%).

Change from Baseline in Outcome Measures

In this study, a change from baseline in outcome measures for the sample overall and by baseline clinical symptomatology was observed (i.e., baseline PHQ-8 or GAD-7 ≥10 vs. baseline PHQ-8 and GAD-7 <10). Significant reductions in perceived stress and burnout over the 9-week intervention period were found among the overall sample, participants with clinically elevated mood symptoms at baseline, and participants without clinically elevated mood symptoms at baseline. Participants with clinically elevated symptoms reported significantly greater reductions in stress compared to those without clinically elevated symptoms. Meanwhile, significant increases in resilience were found over the same period among the overall sample, participants with clinically elevated symptoms, and participants without clinically elevated symptoms. However, there was no statistically significant difference in change of resilience scores between participants with clinically elevated symptoms and those without. Over half of those with burnout at baseline did not continue to have burnout at 9-weeks; no differences in this outcome variable were found for those with versus without clinically elevated symptoms at baseline.

### Bivariate Associations Between Sociodemographic Variables and Outcome Measures

Unadjusted bivariate linear and logistic regression was used to evaluate demographic factors associated with changes in stress and resilience, and burnout at 9 weeks among those with burnout at baseline over the 8-week study period. Results indicate that age was inversely associated with reduced stress such that smaller reductions in stress was observed with increasing age. Females were more likely to continue to have burnout at 9 weeks than males. Participants not currently employed had less decline in stress than those employed full-time. Non-Hispanic Black participants showed a greater decrease in stress and were more likely to show improvement in burnout compared to their non-Hispanic White counterparts. Insured participants had less decline in stress and were less likely to show improvement in burnout than those without insurance. Meanwhile, non-Hispanic Black participants showed less incline in resilience than non-Hispanic White participants, and insured participants had a greater increase in resilience than uninsured.

### Bivariate Associations Between Clinical Characteristics and Outcome Measure

To evaluate clinical factors associated with changes in stress, resilience, and burnout across the intervention period, unadjusted bivariate linear and logistic regression models were estimated. Results show that participants reporting moderate and high levels of stress at baseline showed a greater decrease in stress than those reporting low levels of stress. The magnitude of the decrease in stress was greater for participants with burnout at baseline than those without. Participants with normal baseline levels of resilience showed a greater decrease in stress than those with low levels, while participants with normal to high levels of resilience showed less incline in resilience and were more likely to show improvement in burnout compared to those with low resilience levels. Participants receiving concurrent mental health treatment showed less decline in stress and were less likely to show improvement in burnout than those not receiving treatment. Participants with clinically elevated levels of mood symptoms at baseline showed a significantly greater decrease in stress than those without. No differences in change in resilience or burnout were found between these groups.

### 3.6 Multiple Linear Regression Model of 9-Week Change Scores in Stress, Resilience, and Burnout

Stepwise linear and logistic regression models were estimated to determine whether changes in stress, resilience, and burnout vary by sociodemographic factors, clinical characteristics, and clinical symptomatology at baseline.

To identify variables associated with change in stress, the following variables were retained in the final change in stress model: age; sexual orientation; education level; health insurance; baseline levels of stress, burnout, and resilience; and concurrent mental health treatment. Results indicate that age was inversely associated with reduced stress such that a smaller reduction in stress was observed with increasing age. The magnitude of the decrease in stress also was increased among participants with burnout at baseline than those without. A greater decrease in stress was seen among participants with government-based insurance, moderate and high baseline levels of stress, and normal levels of resilience compared to those without insurance and with low levels of stress and resilience, respectively.

The following variables were retained in the final change in resilience model: age, baseline levels of stress and resilience, and concurrent mental health treatment. Baseline levels of resilience emerged as the only significant predictor of change in resilience in the model: compared to participants with low levels of resilience at baseline, those with normal and high levels showed less increase in resilience across the study period as compared to those with low baseline levels.

Resilience levels at baseline and concurrent mental health treatment comprised the final model to identify factors associated with change in burnout. Results reveal that participants with normal to high levels of resilience were more likely to show improvement in burnout over the study period compared to those with low levels.

### Principal Findings

In this exploratory prospective, single-arm trial of WB-LIFE, we sought to assess the magnitude of change in perceived stress, burnout, and resilience over an 9-week study period as well as explore potential relationships between participant demographic and clinical characteristics and change in each outcome. Exposure to WB-LIFE was associated with significant reductions in perceived stress and burnout and significantly increased resilience over the 9-week study period. A greater reduction in stress was observed among those with clinically elevated mood symptoms (i.e., PHQ-8 or GAD-7 scores ≥10) at baseline compared to those without clinically elevated symptoms; however, differences in the improvement in resilience scores and burnout between the two groups were not statistically significant. While variation in the magnitude of change in stress was observed for participants with and without clinically elevated mood symptoms at baseline, significant improvements in stress, burnout, and resilience over the 9-week study period were observed for both groups.

Baseline levels of the outcome measures were generally associated with the magnitude of 9-week changes in those measures. These findings are consistent with expectations given that greater absolute scores at baseline offer more room for change than smaller scores. Interestingly, participants reporting concurrent mental health treatment experienced smaller reductions in stress and reduced likelihood of showing improvement in burnout than those not reporting concurrent mental health treatment. One potential explanation for these findings is that those receiving concurrent treatment may have previously received therapeutic benefit with respect to the study outcomes, resulting in lower absolute scores at baseline, and thus, less room for change during the study period. Alternatively, those receiving concurrent mental health treatment could be doing so for a condition or concerns that are weakly related to the outcomes of this analysis.

Despite the identification of several significant demographic characteristics in the bivariate analyses, most of the significance was not retained in the multiple regression models that adjusted for baseline levels of each outcome. Baseline resilience was the only factor associated with changes in each of the outcomes in both the bivariate and multivariate analyses, consistent with research showing that resilience is negatively correlated with psychological distress and positively correlated with indicators of psychological well-being 9 (REF). That most of the characteristics found to be significantly associated with the outcomes in the bivariate analysis were not significant in the multivariate analyses that controlled for baseline levels of the outcomes suggests that the benefits of WB-LIFE may extend broadly, rather than being limited to certain subgroups of the population.

Results of this study add to the nascent and growing body of empirical research investigating the impact of mental health relational agents on psychological well-being. Our results are consistent with those of previous studies demonstrating support for mental health conversational agents to reduce stress and depression symptoms in both clinical and non-clinical samples 25 (REF). To date, no studies of which we are aware have investigated the impact of mental health conversational agents on burnout or resilience.

Results of this study must be considered in light of several limitations. First, due to its observational design and lack of a control group, causal links between changes in stress, burnout, and resilience, and the intervention cannot be inferred. Observed changes in outcomes may be attributable to unmeasured factors, such as engagement in other mental health-promoting activities or practices, they may reflect measurement reactivity, or they may be the result of regression to the mean. A randomized controlled trial should be conducted to determine efficacy in improving these outcomes. Second, the extent to which the statistically significant improvements in the outcomes are clinically meaningful is unknown. To our knowledge, there are no accepted minimal clinically important differences (MCIDs), or magnitudes of change that are meaningful to individuals, for the PSS-10, BRS, or the single-item burnout measure evaluated in our study. Third, while our sample was relatively diverse for a naturalistic sample, it was disproportionately female and well-educated, with 69% having college or advanced degrees. Additional studies with more representative samples are warranted to ensure generalizability. Finally, this study examined symptom changes over an 9-week intervention period and did not examine durability of observed changes. In future research, we plan to follow participants beyond the intervention period to investigate longer term outcomes.

Results of this exploratory study suggest that WB-LIFE, a relational agent-guided mental health intervention, may be associated with reduced stress and burnout and increased resilience in populations presenting with clinical and non-clinical levels of mood and anxiety symptoms. Response to such interventions irrespective of baseline clinical symptomatology or baseline demographic or clinical characteristics suggests that delivery to a broad population may be most beneficial to public health. Hypothesis-testing studies are warranted to draw conclusions about efficacy.

1. A method for treating anxiety or depression, the method comprising:
receiving, by a user device and from a therapeutic system, a notification that indicates that a user is to begin a therapeutic session;
triggering, by the user device, a therapeutic session based on the received notification, wherein triggering the therapeutic session includes creating of a messaging session between the user and a conversation engine of the therapeutic system;
aggregating, by the user device, a conversation thread based on communications received from the conversation engine and responses to communications received from the conversation engine input by a user of the user device;
providing, by the user device, response data, to the therapeutic system, that includes the conversation thread;
obtaining, by the user device and from the therapeutic system, therapeutic content based on the response data that, when rendered, by the user device and consumed by a user, treats one or more symptoms of anxiety or depression exhibited by the user; and
administering a therapeutic treatment, by the user device, to the user using the obtained digital content, wherein administering the treatment comprises rendering, by the user device, the obtained digital content on a graphical user interface when the user is viewing the graphical user interface, a predetermined number of times a week for a predetermined duration.

2. The method of claim 1, wherein the conversation thread comprises at least one communication from the conversation engine and at least one communication from the user that is a response to the communication from the conversation engine.

3. The method of claim 1, wherein the conversation thread comprises (i) a single natural language communication from the conversation engine and (ii) a single natural language response from the user.

4. The method of claim 1, wherein the conversation thread comprises (i) multiple natural language communications from the conversation engine and (ii) multiple natural language responses from the user.

5. The method of claim 1, wherein the conversation thread comprises (i) a plurality of pre-programmed graphical elements, with each of the pre-programmed graphical elements corresponding to an emotional state of the user and (ii) response data indicating selection of one or more pre-programmed graphical elements by the user.

6. The method of claim 1, the method further comprising:
receiving, by the user device and from the therapeutic system, a request for feedback following completion of a therapeutic session;
providing, by the user device, feedback data entered by the user to the therapeutic system; and
receiving, by the user device, instructions to terminate the administered treatment prior to the completion of the full duration of the treatment prescribed by the therapeutic system.

7. The method of claim 1, wherein obtaining, by the user device and from the therapeutic system, therapeutic content based on the response data that, when rendered, by the user device and consumed by a user, treats one or more symptoms of anxiety or depression exhibited by the user comprises:
obtaining, by the user device, one or more interpersonal psychotherapy (IPT) tools and one or more cognitive behavioral therapy (CTB) tools based on the response data.

8. The method of claim 1, wherein administering a therapeutic treatment, by the user device, to the user using the obtained digital content, comprises:
rendering, by the user device, one or more interpersonal psychotherapy (IPT) tools and one or more cognitive behavioral therapy (CBT) tools in the user interface of the user device a predetermined number of times a week for a duration of two to eight weeks.

9. The method of claim 1, wherein administering a therapeutic treatment, by the user device, to the user using the obtained digital content comprises:
using the user device to deliver interactive cognitive behavioral therapy (CBT) that comprises natural language interaction with a conversation engine.

10. The method of claim 9, wherein the natural language interaction comprises (i) one or more natural language communications from the conversation engine to the user of the user device and (ii) one or more natural language responses from the user of the user device to the conversation engine.

11. The method of claim 1, wherein administering a therapeutic treatment, by the user device, to the user using the obtained digital content comprises:
using the user device to deliver an interactive therapy that comprises natural language interaction with a conversation engine,
wherein the natural language interaction comprises (i) one or more natural language communications from the conversation engine to the user of the user device and (ii) one or more natural language responses from the user of the user device to the conversation engine.

12. (canceled)

13. (canceled)

14. (canceled)

14. The method of claim 1, wherein the predetermined duration is two to eight weeks.

15. The method of claim 1, wherein the predetermined duration is at least 8 weeks.

16. The method of claim 1, the method for treating anxiety or depression comprises a method for treating clinical levels of anxiety or depression symptoms.

17. The method of claim 1, wherein the therapeutic content is administered for at least 5 minutes during each of the predetermined number of times per week.

18. The method of claim 1, wherein the therapeutic content is administered at least 10 minutes during each of the predetermined number of times per week.

19. The method of claim 1, wherein the predetermined number of times a week for a predetermined duration comprises at least 4 weeks of an 8-week time period.

20. The method of claim 1, wherein anxiety or depression comprises postpartum depression.

21. A system for treating anxiety or depression, the system comprising:
one or more computers; and
one or more storage devices storing instructions that are operable, when executed by the one or more computers, to cause the one or more computers to perform operations, the operations comprising:
receiving, by the one or more computers and from a therapeutic system, a notification that indicates that a user is to begin a therapeutic session;
triggering, by the one or more computers, a therapeutic session based on the received notification, wherein triggering the therapeutic session includes creating of a messaging session between a user of the one or more computers and a conversation engine of the therapeutic system;
aggregating, by the one or more computers, a conversation thread based on communications received from the conversation engine and responses to communications received from the conversation engine input by a user of the one or more computers;
providing, by the one or more computers, response data, to the therapeutic system, that includes the conversation thread;
obtaining, by the one or more computers and from the therapeutic system, therapeutic content based on the response data that, when rendered, by the one or more computers and consumed by a user, treats one or more symptoms of anxiety or depression exhibited by the user; and
administering a therapeutic treatment, by the one or more computers, to the user using the obtained digital content, wherein administering the treatment comprises rendering, by the one or more computers, the obtained digital content on a graphical user interface when the user is viewing the graphical user interface, a predetermined number of times a week for a predetermined duration.

22. The system, of claim 21, wherein the conversation thread comprises at least one communication from the conversation engine and at least one communication from the user that is a response to the communication from the conversation engine.

23. The system of claim 21, wherein the conversation thread comprises (i) a single natural language communication from the conversation engine and (ii) a single natural language response from the user.

24. The system of claim 21, wherein the conversation thread comprises (i) multiple natural language communications from the conversation engine and (ii) multiple natural language responses from the user.

25. The system of claim 21, wherein the conversation thread comprises (i) a plurality of pre-programmed graphical elements, with each of the pre-programmed graphical elements corresponding to an emotional state of the user and (ii) response data indicating selection of one or more pre-programmed graphical elements by the user.

26. The system of claim 21, the operations further comprising:
receiving, by the one or more computers and from the therapeutic system, a request for feedback following completion of a therapeutic session;
providing, by the one or more computers, feedback data entered by the user to the therapeutic system; and
receiving, by the one or more computers, instructions to terminate the administered treatment prior to the completion of the full duration of the treatment prescribed by the therapeutic system.

27. The system of claim 21,
wherein obtaining, by the one or more computers and from the therapeutic system, therapeutic content based on the response data that, when rendered, by the one or more computers and consumed by a user, treats one or more symptoms of anxiety or depression exhibited by the user comprises:
obtaining, by the one or more computers, one or more interpersonal psychotherapy (IPT) tools and one or more cognitive behavioral therapy (CTB) tools based on the response data, and
wherein administering a therapeutic treatment, by the one or more computers, to the user using the obtained digital content, comprises:
rendering, by the one or more computers, one or more interpersonal psychotherapy (IPT) tools and one or more cognitive behavioral therapy (CBT) tools in the user interface of the one or more computers a predetermined number of times a week for a duration of two to eight weeks.

28. The system of claim 21, wherein the predetermined duration is at least 8 weeks.

29. A computer-readable storage medium storing instructions that are operable, when executed by one or more computers, to cause the one or more computers to perform operations for treating anxiety or depression, the operations comprising:
receiving, by the one or more computers and from a therapeutic system, a notification that indicates that a user is to begin a therapeutic session;
triggering, by the one or more computers, a therapeutic session based on the received notification, wherein triggering the therapeutic session includes creating of a messaging session between a user of the one or more computers and a conversation engine of the therapeutic system;
aggregating, by the one or more computers, a conversation thread based on communications received from the conversation engine and responses to communications received from the conversation engine input by a user of the one or more computers;
providing, by the one or more computers, response data, to the therapeutic system, that includes the conversation thread;
obtaining, by the one or more computers and from the therapeutic system, therapeutic content based on the response data that, when rendered, by the one or more computers and consumed by a user, treats one or more symptoms of anxiety or depression exhibited by the user; and
administering a therapeutic treatment, by the one or more computers, to the user using the obtained digital content, wherein administering the treatment comprises rendering, by the one or more computers, the obtained digital content on a graphical user interface when the user is viewing the graphical user interface, a predetermined number of times a week for a predetermined duration.

30. The computer-readable storage medium of claim 29, wherein the predetermined duration is at least 8 weeks.

31. The method of claim 1, wherein the predetermined number of times a week comprises 3 to 5 times per week.

32. The method of claim 1, wherein the predetermined number of times a week comprises daily use during the week.
